SEQUENCE LISTING (2 sequences)

SEQ ID NO: 1 - protein, 44 aa, synthetic construct
  REGION 1..44: note = Lixisenatide
  MOD_RES 44: note = AMIDATION
HGEGTFTSDLSKQMEEEAVRLFIEWLKNGGPSSGAPPSKKKKKK

SEQ ID NO: 2 - protein, 39 aa, synthetic construct
  REGION 1..39: note = Exendin-4
  MOD_RES 39: note = AMIDATION
HGEGTFTSDLSKQMEEEAVRLFIEWLKNGGPSSGAPPPS

(19) United States
(12) Patent Application Publication
(10) Pub. No.: US 2025/0319163 A1
(43) Pub. Date: Oct. 16, 2025

(54) TREATMENT OF TYPE 2 DIABETES MELLITUS

(71) Applicant: SANOFI, Paris (FR)

(72) Inventors: Elisabeth NIEMÖLLER, Frankfurt am Main (DE); Elisabeth SOUHAMI, Paris (FR)

(21) Appl. No.: 19/029,557

(22) Filed: Jan. 17, 2025

Related U.S. Application Data
(63) Continuation of application No. 17/015,857, filed on Sep. 9, 2020, now abandoned.

(30) Foreign Application Priority Data
Sep. 13, 2019 (EP) 19306106.6

(51) Int. Cl. A61K 38/28; A61K 31/155; A61K 31/7048; A61P 3/04

(52) U.S. Cl. CPC A61K 38/28; A61K 31/155; A61K 31/7048; A61P 3/04

(57) ABSTRACT

The present invention relates to a pharmaceutical combination, comprising (a) a pharmaceutical formulation comprising (i) lixisenatide or/and a pharmaceutically acceptable salt thereof, and (ii) insulin glargine or/and a pharmaceutically acceptable salt thereof, and (b) an SGLT2 inhibitor, or/and a pharmaceutically acceptable salt thereof.

### RELATED APPLICATIONS

This application is a continuation of U.S. patent application Ser. No. 17/015,857, filed Sep. 9, 2020, which claims the benefit of European Patent Application No. 19306106.6, filed Sep. 13, 2019, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Jan. 17, 2025, is named 761329_081629-300CON_ST26.xml and is 4,125 bytes in size.

### BRIEF DESCRIPTION OF THE FIGURES

FIG. 1 is a plot of the mean HbA1c (%) by visit during the 26-week randomized treatment period for the mITT population. The plot included all scheduled measurements obtained during the 26-week randomized treatment period, including those obtained after IMP discontinuation or introduction of rescue medication. The number of subjects included for each time point is depicted below the graph. Abbreviations for FIG. 1 are as follows: S=Screening (Week-2), B=Baseline, FRC=Fixed Ratio Combination, GLP-1RA=GLP-1 Receptor Agonist.

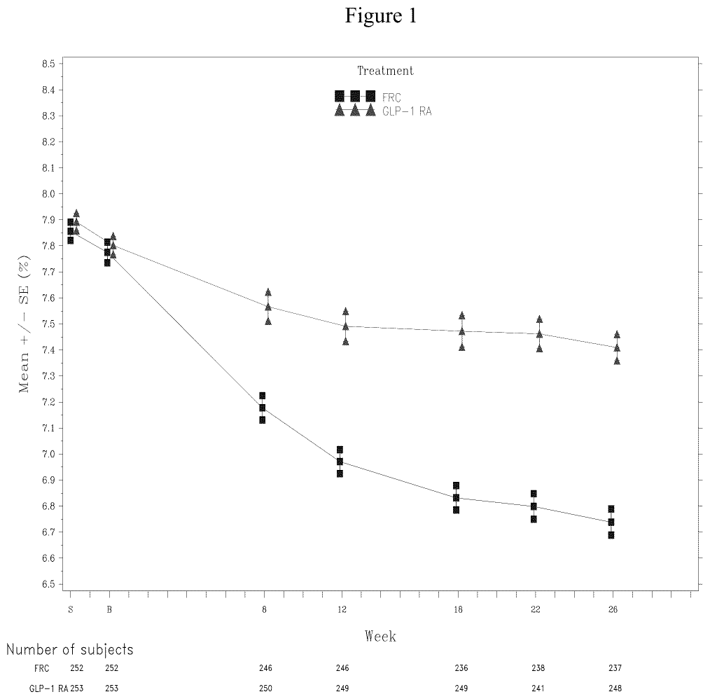

FIG. 2 is a plot of the mean fasting plasma glucose (mmol/L[mg/dL] by visit during the 26-week randomized treatment period—mITT population. The number of subjects included for each time point is depicted below the graph. Abbreviations for FIG. 2 are as follows: S=Screening (Week-2), B=Baseline, FRC=Fixed Ratio Combination, GLP-1RA=GLP-1 Receptor Agonist.

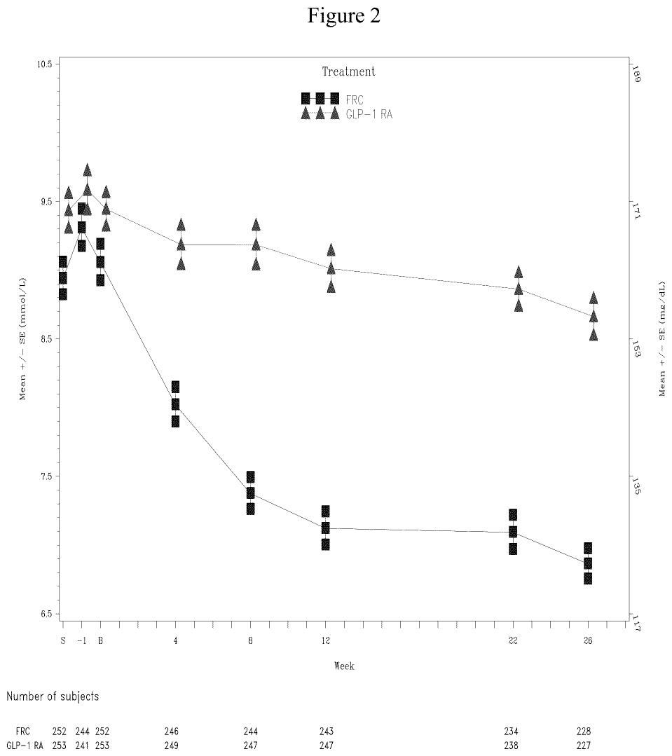

FIG. 3 is a plot of the mean 7-point SMPG (mmol/L[mg/dL]) at baseline and Week 26 during the on-treatment period—mITT population. The number of subjects included for each time point is depicted below the graph. Abbreviations for FIG. 3 are as follows: SMPG=Self-monitored plasma glucose, FRC=Fixed Ratio Combination, GLP-1 RA=GLP-1 Receptor Agonist.

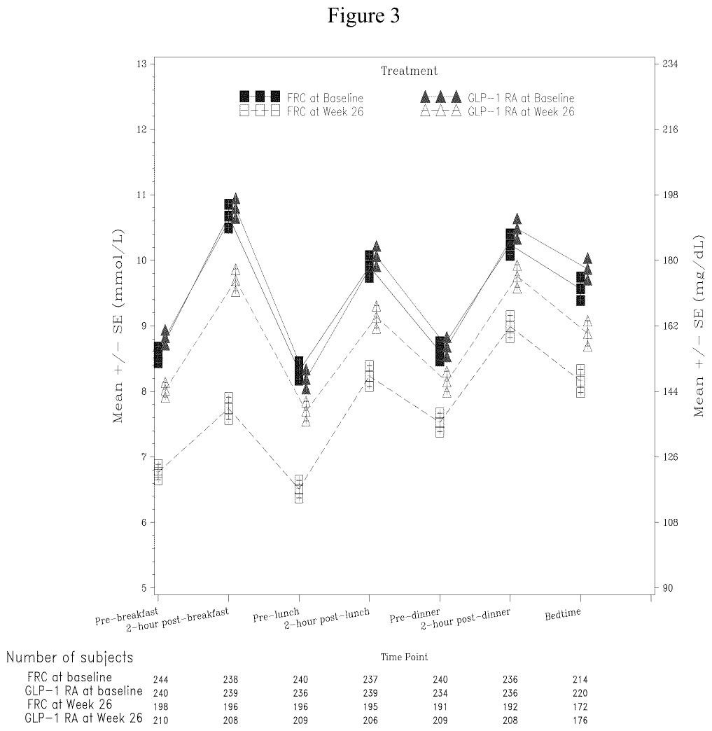

FIG. 4 is graphical diagram of the study design for the clinical trial described in Example 1. Footnotes for FIG. 4 are as follows: (*) additional titration phone calls-only for FRC arm, (**) Insulin glargine/fixed ratio combination (FRC) treatment will be initiated with the Peach Pen. The initial daily dose to be administered will be 10 U: this corresponds to an initial associated dose of insulin glargine 10 U and lixisenatide 5 ug according to the 2/U ug fixed ratio used in the Peach Pen. Afterwards, doses will be individually titrated throughout the study to reach and maintain testing SMPG: 80-100 mg/dL (4.4-6.6 mmol/L) avoiding hypoglycemia. (+) Liraglutide, exenatide, albiglutide, or dulaglutide is discontinued at visit 3 for patients randomized to the FRC treatment. Any patient treated with a once weekly GLP-1 RA upon entering the study who is assigned to received the FRC treatment should not receive their first dose until at least 1 week after their last dose of GLP-1 RA.

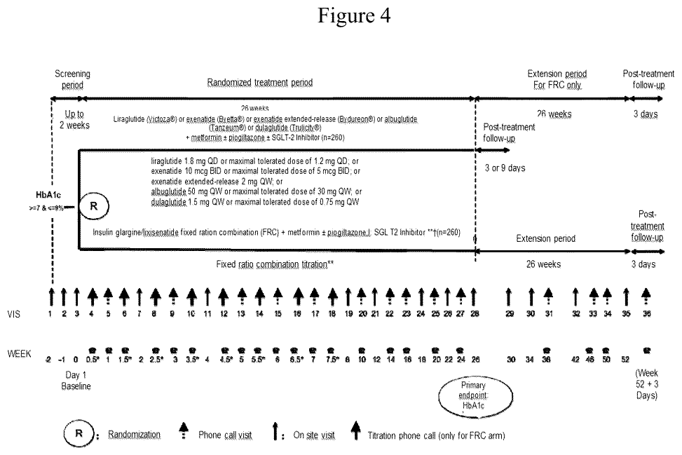

### DESCRIPTION

The present invention relates to a pharmaceutical combination, comprising (a) a pharmaceutical formulation comprising (i) lixisenatide or/and a pharmaceutically acceptable salt thereof, and (ii) insulin glargine or/and a pharmaceutically acceptable salt thereof, and (b) an SGLT2 inhibitor, or/and a pharmaceutically acceptable salt thereof.

Diabetes can be classified into the following general categories (Classification and Diagnosis of Diabetes: Standards of Medical Care in Diabetes-2019—Diabetes Care 2019; 42 (Suppl. 1): S13-S28)
1. Type 1 diabetes (due to autoimmuneb-cell destruction, usually leading to absolute insulin deficiency)
2. Type 2 diabetes (due to a progressive loss of b-cell insulin secretion frequently on the background of insulin resistance)
3. Gestational diabetes mellitus (GDM) (diabetes diagnosed in the second or third trimester of pregnancy that was not clearly overt diabetes prior to gestation)
4. Specific types of diabetes due to other causes, e.g., monogenic diabetes syndromes (such as neonatal diabetes and maturity-onset diabetes of the young [MODY]), diseases of the exocrine pancreas (such as cystic fibrosis and pancreatitis), and drug- or chemical-induced diabetes (such as with glucocorticoid use, in the treatment of HIV/AIDS, or after organ transplantation)

Diabetes may be diagnosed based on plasma glucose criteria, either the fasting plasma glucose (FPG) value or the 2-h plasma glucose (2-h PG) value during a 75-g oral glucose tolerance test (OGTT), or HbAlC criteria

Criteria for the diagnosis of diabetes
FPG≥126 mg/dL (7.0 mmol). Fasting is defined as no caloric intake for at least 8 h.*
or
2-h PG≥200 mg/dL (11.1 mmol/L) during OGTT. The test should be performed as describe by the WHO, using a glucose load containing the equivalent of 75 g anhydrous glucose dissolved in water.*
or
A1C≥6.5% (48 mmol/mol). The test should be performed in a laboratory using a method that is NGSP certified and standardized to the DCCT assay.* or

In a patient with classic symptoms of hyperglycemia or hyperglycemic crisis, a random plasma glucose≥200 mg/dL (11.1 mmol/L).

*In the absence of unequivocal hyperglycemia, diagnosis requires two abnormal test results from the same sample or in two separate test samples.

Type 2 diabetes mellitus is a heterogeneous syndrome characterized by abnormalities in carbohydrate and fat metabolism. The causes of type 2 diabetes are multi-factorial and include both genetic and environmental elements that affect beta-cell function and tissue (muscle, liver, adipose tissue, pancreas) insulin sensitivity (Acta Clin Belg. 2003 November-December; 58(6): 335-41. Pathophysiology of type 2 diabetes. Scheen A J). Normal regulation of glucose metabolism is determined by a feedback loop involving the islet β-cell and insulin-sensitive tissues in which tissue sensitivity to insulin determines the magnitude of the β-cell response. When insulin resistance is present, the β-cell maintains normal glucose tolerance by increasing insulin output. It is only when the β-cell is incapable of releasing sufficient insulin in the presence of insulin resistance that glucose levels rise. While β-cell dysfunction has a clear genetic component, environmental changes play a vital role. (Lancet. 2014 Mar. 22; 383(9922): 1068-1083. Pathophysiology and treatment of type 2 diabetes: perspectives on the past, present and future Steven E. Kahn, M. B., Ch. B., 1 Mark E. Cooper, M. B., B. S, Ph. D., 2 and Stefano Del Prato, M. D. 3).

People with type 2 diabetes are at increased risk of many complications, which are mainly due to complex and inter-connected mechanisms such as hyperglycemia, insulino-resistance, low-grade inflammation and accelerated athero-genesis. Cardio-cerebrovascular disease are frequently associated to type 2 diabetes and may become life threatening, particularly coronaropathy, stroke and heart failure. Type 2 diabetes must be considered as an independent cardiovascular risk factor. Nephropathy is frequent in type 2 diabetes but has a mixed origin. Now it is the highest cause of end-stage renal disease. Better metabolic and blood pressure control and an improved management of microalbuminuria are able to slowdown the course of the disease. Retinopathy which is paradoxically slightly progressive must however be screened and treated in these rather old patients which are globally at high ophthalmologic risk (Jean-Louis Schlienger Presse Med. 2013; 42: 839-848).

A particular risk exists for overweight or obese patients suffering from type 2 diabetes mellitus, e.g. patients with a body mass index (BMI)≥30 kg/m². In these patients the risks of diabetes overlap with the risks of overweight, leading e.g. to an increase of cardiovascular diseases compared to type 2 diabetes mellitus patients being of a normal weight.

Type 2 diabetes is a progressive disease that often requires stepwise intensification of treatment to maintain good glycemic control. It is also well established that timely treatment of people with type 2 diabetes has a beneficial effect on outcomes, so tight glycemic control is advocated to reduce the risk of development or progression of micro or macrovascular complications (Khunti, Diabetes care, 2013)

Progression occurs despite long-term use of standard-of-care oral antidiabetic therapy. Even with the use of multiple oral antidiabetic drugs (OADs), the majority of patients will eventually require the addition of insulin to achieve and maintain HbA1c targets (Khunti as above; Levin P A, Wei W, Zhou S, Xie L, Baser O. Outcomes and treatment patterns of adding a third agent to 2 OADs in patients with type 2 diabetes. J Manag Care Spec Pharm. 2014 May; 20(5): 501-12.).

Patients with T2DM who are inadequately controlled, generally progress stepwise from monotherapy to dual or triple therapy with oral antidiabetic drugs before initiating injectable therapies.

Glucagon-like peptide-1 receptor agonists (GLP-1 RAs) are preferred initial injectable therapy in most patients inadequately controlled on oral therapy, with individualized options for incorporation of basal insulin therapy as required.

Multiple studies have demonstrated the effectiveness of combining a GLP-1 RA with basal insulin as separate injectable therapies administered sequentially (Maiorino M I, Chiodini P, Bellastella G, et al. Free and fixed-ratio combinations of basal insulin and GLP-1 receptor agonists versus basal insulin intensification in type 2 diabetes: a systematic review and meta-analysis of randomized controlled trials. Diabetes Obes Metab 2018; 20:2309-2313; Castellana M, Cignarelli A, Brescia F, Laviola L, Giorgino F. GLP-1 receptor agonist added to insulin versus basal-plus or basal-bolus insulin therapy in type 2 diabetes: a systematic review and meta-analysis. Diabetes Metab Res Rev 2019; 35: e3082). Fixed-ratio combinations (FRCs) of basal insulin plus a GLP-1 RA represent a further advance to facilitate management, with one single injection offering concomitant administration of two effective injectable therapies with complementary modes of action to treat type 2 diabetes.

The compound desPro³⁶Exendin-4(1-39)-Lys₆-NH₂(AVE0010, lixisenatide) is a derivative of Exendin-4. AVE0010 is disclosed as SEQ ID NO:93 in WO 01/04156:

lixisenatide (44 amino acids)
SEQ ID NO: 1:
H-G-E-G-T-F-T-S-D-L-S-K-Q-M-E-E-E-A-V-R-L-F-I-E-W-
L-K-N-G-G-P-S-S-G-A-P-P-S-K-K-K-K-K-K-NH₂
exendin-4 or exenatide (39 amino acids)
SEQ ID NO: 2:
H-G-E-G-T-F-T-S-D-L-S-K-Q-M-E-E-E-A-V-R-L-F-I-E-W-
L-K-N-G-G-P-S-S-G-A-P-P-P-S-NH₂

Exendins are a group of peptides which can lower blood glucose concentration. The Exendin analogue lixisenatide is characterised by C-terminal truncation of the native Exendin-4 sequence. Lixisenatide comprises six C-terminal lysine residues not present in Exendin-4.

Lixisenatide is also termed des-38-proline-exendin-4(Heloderma suspectum)-(1-39)-peptidylpenta-L-lysyl-L-lysinamide (CAS number 320367-13-3). In the present invention, “lixisenatide” includes pharmaceutically acceptable salts thereof. The person skilled in the art knows suitable pharmaceutically acceptable salts of lixisenatide.

Insulin glargine is an analogue of human insulin. Insulin glargine is 31^{B}_32^{B}-Di-Arg human insulin with further substitution of asparagine in position A21 by glycine. Insulin glargine is also termed Gly(A21)-Arg(B31)-Arg(B32) human insulin. The CAS number of insulin glargine is 160337-95-1. In the present invention, “insulin glargine” includes pharmaceutically acceptable salts thereof. The person skilled in the art knows suitable pharmaceutically acceptable salts of insulin glargine. 100 U of insulin glargine correspond to 3.6378 mg of insulin glargine.

Combination formulations of lixisenatide and insulin glargine are disclosed in WO 2014/202483. These formulations contain a fixed-dose ratio of 100 U/mL of insulin glargine and 50 μg/mL of lixisenatide, or 100 U/mL of insulin glargine and 33 μg/mL of lixisenatide. These formulations are marketed under the tradename “Soliqua” or “Suliqua”.

Insulin doses used in Japan are generally lower than those used in Caucasian patients mainly due to lower body mass index (BMI) and insulin resistance of Japanese patients (Møller et al., Diabetes Care. 2014; 37(3): 796-804).

Lixisenatide is approved to be used at the same maintenance dose of 20 μg once daily in the EU, the US and Japan.

Lixisenatide pharmacokinetics (PK) and pharmacodynamics in Caucasian and Japanese patients was assessed in the Phase 1 study PDY6797 (Seino et al., Diabetes Obes Metab. 2014; 16(8): 739-47). Similarity in terms of safety and tolerability between Caucasian and Japanese patients was shown, with a highly overlapping PK profile between the 2 ethnicities. In addition, optimal efficacy with regard to change in postprandial glucose control was observed at the dose level of 20 μg of lixisenatide for both, Caucasian and Japanese patients.

Metformin is the international non-proprietary name of 1,1-dimethylbiguanide (CAS number 657-24-9). Metformin is a biguanide hypoglycemic agent used in the treatment of non-insulin-dependent diabetes mellitus (type 2 diabetes mellitus) not responding to dietary modification. Metformin improves glycemic control by improving insulin sensitivity and decreasing intestinal absorption of glucose. Metformin is usually administered orally. However, control of type 2 diabetes mellitus in obese patients by metformin may be insufficient. Thus, in these patients, additional measures for controlling type 2 diabetes mellitus may be required. “Metformin”, as used herein, includes pharmaceutically acceptable salts thereof. The person skilled in the art knows suitable pharmaceutically acceptable salts of metformin.

Sodium-glucose cotransporter 2 (SGLT2) inhibitors have a mechanism of action, which is independent of insulin secretion and insulin action. By inhibiting SGLT2 in the renal proximal tubule, they reduce renal glucose reabsorption, causing urinary glucose excretion and thereby lower plasma glucose. This unique mechanism of action, in addition to lowering plasma glucose, corrects a number of metabolic and hemodynamic abnormalities that are risk factors for cardiovascular diseases (Abdul-Ghani M A, et al., Endocr Rev 2011; 32: 515-531, Abdul-Ghani M A, et al., Diabetes Care 2016; 39:717-725). Urinary glucose loss produces negative caloric balance, resulting in weight loss. SGLT2 inhibition decreases sodium reabsorption in the proximal tubule and exerts diuretic/natriuretic effects (Lambers et al., Diabetes Obes Metab 2013; 15:853-862). SGLT2 inhibition also promotes urinary sodium excretion by causing osmotic diuresis. This natriuretic effect, combined with the more long-term reduction in body weight, contributes, in part, to decreases in systolic/diastolic blood pressure (Abdul-Ghani et al., Am J Physiol Renal Physiol 2015; 309: F889-F900).

SGLT2 inhibitors are usually administered orally. However, control of type 2 diabetes mellitus in obese patients by SGLT2 inhibitors may be insufficient. Thus, in these patients, additional measures for controlling type 2 diabetes mellitus may be required.

An SGLT2 inhibitor alone may be insufficient to achieve adequate glycemic control. Also an SGLT2 inhibitor, combined with a second antidiabetic, such as metformin or/and a GLP-1 receptor agonist, may be insufficient. Therefore there is a need of a suitable treatment regimen in these patients. The problem of the invention can be seen in the provision of a suitable treatment regimen in these patients.

The addition of a second or third anti-diabetic poses the problem that undesired side effects may occur.

In the Examples of the invention, it is demonstrated that in type 2 diabetes patients, receiving an SGLT2 inhibitor, being insufficient to achieve adequate glycemic control, the addition of a fixed-dose ratio formulation of insulin glargine and lixisenatide can improve glycemic control, with an improved side effect profile.

A first aspect of the present invention is a pharmaceutical combination, comprising
(a) a pharmaceutical formulation comprising
   (i) lixisenatide or/and a pharmaceutically acceptable salt thereof, and
   (ii) insulin glargine or/and a pharmaceutically acceptable salt thereof, and
(b) an SGLT2 inhibitor, or/and a pharmaceutically acceptable salt thereof.

In the present invention, it has been found that the combination of a fixed-dose ratio formulation of lixisenatide and insulin glargine, combined with an SGLT-2 inhibitor, is efficacious and safe in view of a fixed-dose ratio formulation of lixisenatide and insulin glargine not combined with an SGLT2 inhibitor. In the trials described herein, in total 119 type 2 diabetes mellitus patients received an insulin glargine/lixisenatide fixed ratio combination (FRC) and also received an SGLT2 inhibitor.

As insulin doses used in Japan are generally lower than those used in Caucasian patients mainly due to lower body mass index (BMI) and insulin resistance of Japanese patients (M¥ller et al., Diabetes Care. 2014: 37(3): 796-804), in the Examples of the invention, different fixed ratios of the combination are used. In Examples 1 and 4, including patients of 9 countries (Canada, Estonia, Germany, Israel, Italy, Romania, Slovakia, Spain, United States), being considered as “Caucasian” patients, fixed-dose ratio formulations of 2 units insulin glargine U100 (i.e. 100 U/ml) per 1 μg lixisenatide and 3 units insulin glargine U100 per 1 μg lixisenatide are used. In Examples 2, 3, 5 and 6, including Japanese patients, a fixed-dose ratio formulation comprising 1 unit insulin glargine U100 per 1 μg lixisenatide is used. These formulations offer an appropriate dose range, 10 to 60 units in the Examples 1 and 4, and 5 to 20 units in Examples 2, 3, 5 and 6, covering the need of the vast majority of patients in each of the two populations.

Example 1 relates to a study assessing the efficacy and safety of the insulin glargine/lixisenatide fixed ratio combination in adults with Type 2 Diabetes inadequately controlled on GLP-1 receptor agonist and metformin (alone or with pioglitazone and/or SGLT2 inhibitors), followed by a fixed ratio combination single-arm 26-week extension period (Study EFC 13794).

In Example 1, the investigational treatment included a fixed dose ratio formulation comprising 100 U/mL of insulin glargine and 50 μg/mL of lixisenatide, or comprising 100 U/mL of insulin glargine and 33 μg/mL of lixisenatide, on top of metformin with or without the SGLT2 inhibitor. The investigational treatment was compared with the continuation of the GLP-1 receptor agonist as active comparator, on top of metformin with or without an SGLT2 inhibitor.

Example 4 summarizes a sub-group analysis of the data of example 1, comparing patients receiving an SGLT2 inhibitor with patients not receiving an SGLT2 inhibitor. Type 2 diabetes patients were included receiving metformin, a GLP-1 receptor agonist selected from liraglutide, exenatide, an exenatide extended release formulation, albiglutide and dulaglutide, with or without an SGLT2 inhibitor. An improvement was observed in efficacy results (change from baseline to Week 26 in glycated hemoglobin [HbA1c], fasting plasma glucose [FPG] and 2-hour postprandial plasma glucose [PPG]) in both treatment groups.

According to Example 4, in patients receiving an SGLT2 inhibitor, the effect of the fixed-ratio formulation was larger than in the comparative treatment, continuing GLP-1 RA, compared with the patient group not receiving an SGLT2 inhibitor.

In patients receiving an SGLT2 inhibitor, after 26 weeks improvement in HbA1c was 0.88% in view of the active comparator, compared with 0.61% in patients not receiving an SGLT2 inhibitor (Table 3 of Example 4).

In patients receiving an SGLT2 inhibitor, after 26 weeks, improvement in fasting plasma glucose was 2.06 mmol/L in view of the active comparator, compared with 1.64 mmol/L in patients not receiving an SGLT2 inhibitor (Table 4 of Example 4).

In patients receiving an SGLT2 inhibitor, after 26 weeks, improvement in 2 hour postprandial glucose was 3.26 mmol/L in view of the active comparator, compared with 2.81 mmol/L in patients not receiving an SGLT2 inhibitor (Table 5 of Example 4).

In Example 4 (Table 8) documented symptomatic hypoglycemia (plasma glucose ≤3.9 mmol/L [≤70 mg/dL]) was reported less frequently in the FRC group using SGLT2i versus non-users (0.72 events per patient year for SGLT2i users versus 1.62 for non-users).

In conclusion, Example 4 demonstrates that in patients, using an SGLT2 inhibitor in combination with a fixed-dose ratio formulation of insulin glargine and lixisenatide, an improved glycemic control and an improved side effect profile can be achieved, compared with patients not using an SGLT2 inhibitor.

Examples 2 and 3 refer to clinical trials in Japanese patients, receiving a fixed dose ratio formulation comprising 100 U/mL of insulin glargine and 100 μg/mL of lixisenatide.

Example 2 relates to a study comparing the efficacy and safety of the insulin glargine/lixisenatide fixed-ratio combination to lixisenatide in combination with oral antidiabetic drugs in Japanese patients with type 2 diabetes mellitus inadequately controlled on oral antidiabetic drugs, with a 26-week safety extension period.

Example 3 relates to a study comparing the efficacy and safety of the insulin glargine/lixisenatide fixed-ratio combination to insulin glargine in combination with oral antidiabetic drugs in Japanese patients with type 2 diabetes mellitus inadequately controlled on oral antidiabetic drugs.

In Examples 2 and 3, one or two of the following oral antidiabetic drugs was allowed to be used as background therapy during the study: biguanide (for example metformin), thiazolidinedione (TZD), alpha-glucosidase inhibitor (alpha-GI), SGLT2 inhibitor, glinide, and sulfonylurea (SU).

Example 5 summarizes a sub-group analysis of the data of example 2, comparing patient receiving an SGLT2 inhibitor with patients not receiving an SGLT2 inhibitor. Efficacy results (change from baseline to Week 26 in HbA1c and FPG) in both treatment groups were generally similar in SGLT2i users and non-users (Table 3, Table 4 of Example 5).

With regard to common treatment-emergent adverse events (TEAEs) (Table 6 of Example 5), TEAEs in the gastrointestinal disorder System Organ Class (SOC) were reported less frequently in the FRC treatment group in SGLT2i users when compared to the SGLT2i non-users (17.6% versus 32.3%, respectively). Similarly, documented symptomatic hypoglycemia (plasma glucose ≤3.9 mmol/L [≤70 mg/dL]) in the FRC group was reported less frequently in SGLT2i users compared to non-users (number of events per patient year: 0.18 and 1.14, respectively) (Table 7 of Example 5).

Example 6 summarizes a sub-group analysis of the data of example 3, comparing patient receiving an SGLT2 inhibitor with patients not receiving an SGLT2 inhibitor. Efficacy results (change from baseline to Week 26 in HbA1c, FPG and 2-hour PPG) in both treatment groups were generally similar in SGLT2i users and non-users (Table 3, Table 4, Table 5 of Example 6). There was no indication of a decreased efficacy of the FRC in the SGLT2i user subgroup.

With regard to common TEAEs (Table 7 of Example 6), TEAEs in the gastrointestinal System Organ Class (SOC) were also reported numerically less frequently in the FRC group in SGLT2i users when compared to SGLT2i non-users (22.0% versus 27.4%, respectively). Documented symptomatic hypoglycemia (plasma glucose ≤3.9 mmol/L) was reported in similar proportions of SGLT2i users and non-users (Table 8 of Example 6).

In conclusion, Examples 5 and 6 demonstrate that in patients, using an SGLT2 inhibitor in combination with a fixed-dose ratio formulation of insulin glargine and lixisenatide, an improved side effect profile can be achieved, compared with patients not using an SGLT2 inhibitor.

“SGLT2 inhibitor”, as used herein, includes pharmaceutically acceptable salts thereof. The person skilled in the art knows suitable pharmaceutically acceptable salts of SGLT2 inhibitors. “SGLT2 inhibitor” are also termed herein as “SGLT-2 inhibitor” or “SGLT2i”.

In the present invention, the SGLT2 inhibitor can be selected from the group consisting of empagliflozin, canagliflozin, dapagliflozin and ertugliflozin. For example, the SGLT2 inhibitor can be selected from empagliflozin, canagliflozin and dapagliflozin.

The skilled person knows suitable doses of the SGLT2 inhibitor to be administered. In the combination of the present invention, canagliflozin can be administered in a daily dose in the range of 100 to 300 mg. In the combination of the present invention, empagliflozin can be administered in a daily dose in the range of 10 to 25 mg. In the combination of the present invention, dapagliflozin can be administered in a daily dose in the range of 5 to 20 mg.

In the pharmaceutical combination of the present invention the pharmaceutical formulation (a) can comprise insulin glargine in a concentration of 100 to 500 U/mL. For example, the pharmaceutical formulation (a) can comprise insulin glargine in a concentration of 100 U/mL.

In the pharmaceutical combination of the present invention the pharmaceutical formulation (a) can comprise lixisenatide in a concentration of 20 to 150 μg/ml. For example, the pharmaceutical formulation (a) can comprise lixisenatide in a concentration of 33 μg/mL, 50 μg/mL or 100 μg/mL.

In another aspect of the present invention, the pharmaceutical formulation (a) comprises insulin glargine in a concentration of 100 U/mL and lixisenatide in a concentration of 33 μg/mL, 50 μg/mL or 100 μg/mL.

The pharmaceutical combination of the present invention can be used for the treatment of type 2 diabetes mellitus, for example in a human patient.

The patient may be a Caucasian patient, or may be an Asian patient, for example a Chinese or a Japanese patient.

The formulation (a) can comprise insulin glargine in a concentration of 100 U/mL and lixisenatide in a concentration of 33 μg/mL or 50 μg/mL. This formulation is suitable in the treatment of a Caucasian type 2 diabetes mellitus patient, but the use of this formulation is not limited to this patient group.

The formulation (a) may comprise insulin glargine in a concentration of 100 U/mL and lixisenatide in a concentration of 100 μg/mL. This formulation is suitable in the treatment of an Asian type 2 diabetes mellitus patient, for example a Chinese or a Japanese patient, but the use of this formulation is not limited to this patient group.

As demonstrated by the Examples of the invention, the combination as described herein can be used for improving glycemic control in type 2 diabetes mellitus patients. In the present invention, “improvement of glycemic control” or “glycemic control” for example refers to improvement of the 2 hour postprandial plasma glucose concentration, improvement of fasting plasma glucose concentration, improvement of self-monitored plasma glucose (SMPG) or/and improvement of the HbA_{1c} value.

For example, “improvement of glycemic control” or “glycemic control” can include the improvement of the 2 hour postprandial plasma glucose concentration.

For example, “improvement of glycemic control” or “glycemic control” can include the reduction of the 2 hour postprandial plasma glucose concentration. Reduction means for example that the 2 hour postprandial plasma glucose concentration reaches normoglycemic values or at least approaches these values.

For example, “improvement of glycemic control” or “glycemic control” can include the improvement of the fasting plasma glucose concentration.

For example, improvement of fasting plasma glucose concentration can include the reduction of the fasting plasma glucose concentration. Reduction means for example that the fasting plasma glucose concentration reaches normoglycemic values or at least approaches these values.

For example, “improvement of glycemic control” or “glycemic control” can include the improvement of the self-monitored glucose concentration.

For example, improvement of self-monitored glucose concentration can include the reduction of the self-monitored glucose concentration. Reduction means for example that the self-monitored glucose concentration reaches normoglycemic values or at least approaches these values.

For example, “improvement of glycemic control” or “glycemic control” can include the improvement of the HbA_{1c} value.

For example, improvement of the HbA_{1c} value can include the reduction of the HbA_{1c} value. Reduction of the HbA_{1c} value for example means that the HbA_{1c} value is reduced below 6.5% or 7%.

In the present invention, normoglycemic values of fasting plasma glucose are blood glucose concentrations of for example <5.6 mmol/L.

In the present invention, normoglycemic values of postprandial plasma glucose, as defined herein, are blood glucose concentrations of for example <7.8 mmol/L.

In the present invention, normoglycemic HbA1c values are for example <6.5% or <7%.

In another aspect of the present invention, the type 2 diabetes mellitus to be treated is not adequately controlled with an SGLT2 inhibitor alone, for example with empagliflozin, canagliflozin, dapagliflozin or ertugliflozin alone.

As used herein, the term “oral anti-diabetic” includes biguanides, thiazolidinediones, alpha-glucosidase inhibitors, glinides, and sulfonylureas, but is not limited to these compounds. For example the biguanide is metformin.

As used herein, the term “GLP-1 receptor agonist” or “GLP-1 RA” includes lixisenatide, exenatide, dulaglutide, liraglutide, and albiglutide, but is not limited to these compounds. Exenatide can also be administered in an extended-release formulation.

In another aspect, the type 2 diabetes mellitus to be treated is not adequately controlled with an SGLT2 inhibitor and an oral anti-diabetic alone, or with an SGLT2 inhibitor and a GLP-1 receptor agonist alone. The oral anti-diabetic may be selected from the group of biguanides, thiazolidinediones, alpha-glucosidase inhibitors, glinides, and sulfonylureas. For example the biguanide is metformin.

In another aspect, the type 2 diabetes mellitus to be treated is not adequately controlled (i) with an SGLT2 inhibitor and metformin alone, or (ii) with an SGLT2 inhibitor and a GLP-1 receptor agonist alone.

In yet another aspect, the type 2 diabetes mellitus to be treated is not adequately controlled with the SGLT2 inhibitor, metformin and a GLP-1 receptor agonist alone.

In the present invention, “not adequately controlled” by an anti-diabetic treatment means that this treatment is not sufficient to remove the symptoms of type 2 diabetes mellitus. For example, “not adequately controlled” by this treatment means that the patient does not reach normoglycemic values in terms of, for example, 2 hour postprandial plasma glucose concentration, SMPG, HbA_{1c} value or/and fasting plasma glucose concentration. For example, the anti-diabetic pre-treatment is insufficient to achieve adequate glycemic control.

In the present invention, “pre-treatment”, “treatment prior to administration of the combination of the invention”, or treatment of “the patient to be treated according to the invention” relates to the anti-diabetic treatment which the patient receives before receiving the combination of the invention, for example within one, two, three months or within a longer period, before receiving the combination of the invention.

As used herein, “to be treated according to the present invention”, “treatment according to the present invention”, or “therapy according to the present invention” relates to the treatment of a type 2 diabetes mellitus patient by the pharmaceutical combination of the invention.

Metformin being used in the treatment prior to administration of the combination of the invention can be administered for instance in a dose of at least 1.0 g/day metformin or at least 1.5 g/day metformin for at least 3 months, or/and in a dose of at the maximum 2.0 g/day metformin for at least 3 months or at the maximum 3.5 g/day metformin for at least 3 months. The daily dose may also be in the range of 500 to 3000 mg, for example 1000 to 2600 mg.

The GLP-1 receptor agonist being used in the treatment prior to administration of the combination of the invention can be selected from lixisenatide, exenatide, dulaglutide, liraglutide, and albiglutide. Exenatide can also be administered in an extended-release formulation.

For example, the GLP-1 receptor agonist being used in the treatment prior to administration of the combination of the invention can be selected from lixisenatide, exenatide, dulaglutide, and liraglutide.

Formulations comprising a GLP-1 receptor agonist, such as selected from lixisenatide, exenatide, dulaglutide, liraglutide and albiglutide are known to the skilled person.

Typical doses of GLP-1 receptor agonists are known to the skilled person. In the present invention, the daily dose of exenatide can be in the range of 10-20 μg. The weekly dose of exenatide in an extended-release formulation can be 2 mg. The daily dose of dulaglutide can be in the range of 0.75-1.5 mg. The daily dose of liraglutide can be in the range of 1.2-1.8 mg. The daily dose of albiglutide can be in the range of 30 to 50 mg. The daily dose of lixisenatide can be in the range of 10-20 μg.

A typical daily dose of insulin glargine, to be administered with the pharmaceutical formulation (a) of the present invention, is 5 to 60 U, and the corresponding dose of lixisenatide. For example, in a formulation comprising 100 U/mL of insulin glargine and 50 μg/mL of lixisenatide, this dosage range corresponds to a daily dose of lixisenatide of 2.5 to 30 μg. For example, in a formulation comprising 100 U/mL of insulin glargine and 33 μg/mL of lixisenatide, this dosage range corresponds to a daily dose of lixisenatide of about 1.6 to 20 μg.

For example, a dose of 10 to 60 U can be administered. This dosage range is suitable in the treatment of a Caucasian type 2 diabetes mellitus patient, but the use of this dosage is not limited to this patient group. For example, in a formulation comprising 100 U/mL of insulin glargine and 50 μg/mL of lixisenatide, this dosage range corresponds to a daily dose of lixisenatide of 5 to 30 μg. For example, in a formulation comprising 100 U/mL of insulin glargine and 33 μg/mL of lixisenatide, this dosage range corresponds to a daily dose of lixisenatide of about 3.3 to 20 μg.

For example, a dose of 5 to 20 U can be administered. This dosage range is suitable in the treatment of an Asian type 2 diabetes mellitus patient, for example a Japanese or Chinese patient, but the use of this dosage is not limited to this patient group. For example, in a formulation comprising 100 U/mL of insulin glargine and 100 μg/mL of lixisenatide, this dosage range corresponds to a daily dose of lixisenatide of 5 to 20 μg.

The pre-treatment, being insufficient to adequate control the type 2 diabetes, as described herein, may also include the treatment with pioglitazone. Formulations comprising pioglitazone being used in the treatment prior to administration of the combination of the invention are known to the skilled person.

In the present invention, the daily dose of pioglitazone can be in the range of 15 to 45 mg, for example 30 mg.

The type 2 diabetes mellitus patient suffering from type 2 diabetes mellitus to be treated according to the present invention may be obese. A patient can be considered as obese if the body mass index is at least 30 kg/m² (Caucasian patient) or at least 25 kg/m² (Asian patient, for example a Chinese or a Japanese patient).

In the present invention, an obese type 2 diabetes mellitus patient may have a body mass index of at least 30 kg/m² at least 31 kg/m² or at least 32 kg/m², which for example is a Caucasian patient. If the patient is an Asian patient, for example a Chinese or a Japanese patient, the patient may have a body mass index of at least 25 kg/m² or at least 26 kg/m². The type 2 diabetes mellitus patient may be obese prior to the onset of therapy with the combination according to the present invention.

The patient to be treated may have an age of less than 50 years. The patient may also have an age of at least 50 years.

In the present invention, the type 2 diabetes mellitus patient may have a HbA_{1c} value in the range of 7% to 9%, in the range of 7.5% to 9.5%, or in the range of 7.5% to 10%. The type 2 diabetes mellitus patient may have a HbA_{1c} of at least 7.5%, at least 7.8%, or at least 8%. These HbA_{1c} values exceed normoglycemic values, indicating that the type 2 diabetes mellitus is not adequately controlled if treated with an antidiabetic compound.

Prior to the onset of therapy with the combination according to the present invention, the patient may have a HbA1c as described herein, for example of at least 7.5%, at least 7.8%, or at least 8% when treated with
(a) an SGLT2 inhibitor alone,
(b) an SGLT2 inhibitor in combination with an oral antidiabetic, as described herein,
(c) an SGLT2 inhibitor in combination with metformin, or
(d) an SGLT2 inhibitor in combination with metformin and a GLP-1 receptor agonist, as described herein.

The oral anti-diabetic treatment may be selected from the group of biguanides, thiazolidinediones, alpha-glucosidase inhibitors, glinides, and sulfonylureas. For example the biguanide is metformin, as described herein. The GLP-1 receptor agonist may be selected from liraglutide, lixisenatide, exenatide, an exenatide extended release formulation, albiglutide and dulaglutide, as described herein.

The type 2 diabetes mellitus patient to be treated according to the invention may have a fasting plasma glucose concentration of at least 8 mmol/L, at least 8.5 mmol/L, or at least 9 mmol/L. These plasma glucose concentrations exceed normoglycemic concentrations, indicating that the type 2 diabetes mellitus is not adequately controlled if treated with an antidiabetic compound.

Prior to the onset of therapy with the combination according to the present invention, the patient may have a fasting plasma glucose of HbA1c of at least 8 mmol/L, at least 8.5 mmol/L, or at least 9 mmol/L when treated with
(a) an SGLT2 inhibitor alone,
(b) an SGLT2 inhibitor in combination with an oral antidiabetic, as described herein,
(c) an SGLT2 inhibitor in combination with metformin, or
(d) an SGLT2 inhibitor in combination with metformin and a GLP-1 receptor agonist, as described herein.

The oral anti-diabetic treatment may be selected from the group of biguanides, thiazolidinediones, alpha-glucosidase inhibitors, glinides, and sulfonylureas. For example, the biguanide is metformin. The GLP-1 receptor agonist may be selected from liraglutide, lixisenatide, exenatide, an exenatide extended release formulation, albiglutide and dulaglutide, as described herein.

For example, in the patient to be treated according to the present invention, the type 2 diabetes mellitus has been diagnosed for at least 1 year or at least 2 years prior to the onset of a therapy according to the present invention.

“Self-monitored plasma glucose (SMPG)”, as used herein, can be the “4-point Self Monitored Plasma Glucose” or the “7-point Self Monitored Plasma Glucose”. The 4 point and 7-point Self Monitored Plasma Glucose value are for example average plasma glucose concentrations including fasting and postprandial conditions.

“4-point Self Monitored Plasma Glucose” for example refers to the measurement of plasma glucose four times a day and calculation of the average plasma glucose concentration therefrom. For example, the 4-point Self Monitored Plasma Glucose measurements are performed pre-breakfast, post-breakfast, pre-dinner, and post-dinner.

“7-point Self Monitored Plasma Glucose” for example refers to the measurement of plasma glucose seven times a day and calculation of the average plasma glucose concentration therefrom. For example, the 7-point Self Monitored Plasma Glucose measurements are performed pre-breakfast, post-breakfast, pre-lunch, post-lunch, pre-dinner, post-dinner and at bed-time.

The “fasting self-monitored plasma glucose (SMPG)”, as used herein, is measured by the patient before breakfast, for example before insulin glargine or/and lixisenatide injection and optional intake of metformin.

The type 2 diabetes mellitus patient to be treated according to the present invention may have a 2 hours postprandial plasma glucose concentration of at least 11.1 mmol/L. These plasma glucose concentrations exceed normoglycemic concentrations, indicating that the type 2 diabetes mellitus is not adequately controlled if treated with an antidiabetic compound.

“Postprandial” is a term that is well known to a person skilled in the art of diabetology. The term “postprandial” describes for example the phase after an ingestion of a meal or/and exposure to glucose under experimental conditions. In a healthy person this phase is characterised by an increase and subsequent decrease in blood glucose concentration. The postprandial phase typically ends up to 2 h after a meal or/and exposure to glucose (2 h postprandial plasma glucose concentration).

Determination of postprandial plasma glucose is well-known (see, e.g. Crapo et al., Diabetes, 1977, 26(12):1178-1183).

As disclosed herein the patient can be an Asian patient, for example a Chinese or a Japanese patient. For example, the Asian patient, for example a Chinese or a Japanese patient, may be obese. The Asian patient, for example a Chinese or a Japanese patient may have a body mass index of at least 25 kg/m² or at least 26 kg/m². The Asian patient, for example a Chinese or a Japanese patient may be obese prior to the onset of therapy with the combination according to the present invention.

The formulation (a) of the present invention may comprise insulin glargine in a concentration of 100 U/mL and lixisenatide in a concentration of 100 μg/mL, when used in an Asian patient, for example a Chinese or a Japanese patient.

In another aspect of the invention, in the Asian patient, for example a Chinese or a Japanese patient, the type 2 diabetes mellitus to be treated is not adequately controlled with an SGLT2 inhibitor and an oral anti-diabetic alone. The oral anti-diabetic may be selected from the group of biguanides, thiazolidinediones, alpha-glucosidase inhibitors, glinides, and sulfonylureas. For example the biguanide is metformin.

In the present invention, metformin can be administered according to commonly known administration protocols of metformin in accordance with the terms of marketing authorization. The skilled person knows suitable dosage forms. For example, metformin can be administrated once daily, twice daily or three times a day. For example, the metformin dose applied before the onset of the therapy as disclosed herein is continued with the treatment of the invention, as disclosed herein.

In the present invention, metformin may be administered orally. For oral administration, metformin may be formulated in a solid dosage form, such as a tablet or pill. Metformin may be formulated with suitable pharmaceutically acceptable carriers, adjuvants, or/and auxiliary substances.

In the present invention, the SGLT-2 inhibitor can be administered according to commonly known administration protocols of the SGLT-2 inhibitor in accordance with the terms of marketing authorization. The skilled person knows suitable dosage forms. For example, the SGLT-2 inhibitor can be administrated once daily, twice daily or three times a day. For example, the SGLT-2 inhibitor dose applied before the onset of the therapy as disclosed herein is continued with the treatment of the invention, as disclosed herein.

In the present invention, the SGLT-2 inhibitor may be administered orally. For oral administration, the SGLT-2 inhibitor may be formulated in a solid dosage form, such as a tablet or pill. The SGLT-2 inhibitor may be formulated with suitable pharmaceutically acceptable carriers, adjuvants, or/and auxiliary substances.

The pharmaceutical combination of the invention allows a simultaneous, separate or sequential administration of (a) the pharmaceutical formulation comprising lixisenatide or/and a pharmaceutically acceptable salt thereof and insulin glargine or/and a pharmaceutically acceptable salt thereof, and (b) of the SGLT2 inhibitor or/and a pharmaceutically acceptable salt thereof and, optionally, (c) of metformin or/and a pharmaceutically acceptable salt thereof.

In the present invention “separate administration” means that the pharmaceutical combination according to the invention may be administered in separate pharmaceutical formulations, wherein one pharmaceutical formulation (a) comprises lixisenatide or/and a pharmaceutically acceptable salt thereof, and insulin glargine or/and a pharmaceutically acceptable salt thereof, and the second pharmaceutical formulation (b) comprises an SGLT2 inhibitor, or/and a pharmaceutically acceptable salt thereof. Optionally, a third pharmaceutical formulation (c) comprising metformin, or/and a pharmaceutically acceptable salt thereof, may be administered.

The pharmaceutical formulations can be administered simultaneously or successively in any sequence. The invention thus relates, for example, to a pharmaceutical combination comprising an injectable pharmaceutical formulation comprising lixisenatide or/and a pharmaceutically acceptable salt thereof and insulin glargine or/and a pharmaceutically acceptable salt thereof, and an oral pharmaceutical formulation comprising an SGLT2 inhibitor, or/and a pharmaceutically acceptable salt thereof, and optionally an oral pharmaceutical formulation comprising metformin or/and a pharmaceutically acceptable salt thereof by simultaneous, separate or sequential administration, in particular for use in the treatment of a type 2 diabetes mellitus patient.

In the present invention, simultaneous administration may include administration of the formulations of the invention at the same time, or within a time interval necessary to administer the compositions of the invention, for example within 5 min, 10 min, or 15 min.

In the present invention, sequential administration may include administration of the formulations of the invention at intervals of at least 15 min, at least 1 h, or at least 2 h, or at intervals of up to 3 h. If optionally a formulation comprising metformin or/and a pharmaceutically acceptable salt thereof, is administered, the intervals between administration of the formulations may be selected independently. Any sequence of administration may be selected. For example, administration can start with (a) the pharmaceutical formulation comprising lixisenatide or/and a pharmaceutically acceptable salt thereof and insulin glargine or/and a pharmaceutically acceptable salt thereof, and can continue with the formulation (b) comprising the SGLT2 inhibitor or/and a pharmaceutically acceptable salt thereof, or vice versa. If optionally a formulation (c) comprising metformin or/and a pharmaceutically acceptable salt thereof, is administered, this formulation may be administered before, in between or after formulations (a) and (b). For example, administration may start with the formulation (c) comprising metformin or/and the pharmaceutically acceptable salt thereof, and may continue with (a) the pharmaceutical formulation comprising lixisenatide or/and a pharmaceutically acceptable salt thereof and insulin glargine or/and a pharmaceutically acceptable salt thereof, and then may continue with the formulation (b) comprising the SGLT2 inhibitor or/and a pharmaceutically acceptable salt thereof, or vice versa.

As disclosed herein the formulation comprising lixisenatide or/and a pharmaceutically acceptable salt thereof and insulin glargine or/and a pharmaceutically acceptable salt thereof, may be administered by one injection per day. If, for example, the oral formulation comprising the SGLT2 inhibitor or/and a pharmaceutically acceptable salt thereof, is administered more than once daily, for example twice daily or three times daily, one of the doses can be administered simultaneously with the formulation comprising lixisenatide or/and a pharmaceutically acceptable salt thereof and insulin glargine or/and a pharmaceutically acceptable salt thereof. If, for example, the optional oral formulation comprising metformin or/and a pharmaceutically acceptable salt thereof, is administered more than once daily, for example twice daily or three times daily, one of the doses can be administered simultaneously with the formulation comprising lixisenatide or/and a pharmaceutically acceptable salt thereof and insulin glargine or/and a pharmaceutically acceptable salt thereof.

In the present invention, the pharmaceutical combination, as described herein, may be administered in an add-on therapy.

In the present invention, the terms “add-on”, “add-on treatment” and “add-on therapy” relate to treatment according to the present invention, wherein the pre-treatment with at least one anti-diabetic is continued. “Add-on”, “add-on treatment” and “add-on therapy” for example mean that the dose of the at least one anti-diabetic administered before the onset of the treatment according to the present invention, as disclosed herein, can be continued in the treatment of the present invention. If the pre-treatment includes the administration of an SGLT2 inhibitor, in an add-on therapy, the treatment with the SGLT2 inhibitor is continued, for example with the same dose. If necessary, the dose or the doses of one or more of the at least one anti-diabetic administered in the pre-treatment regimen can be adapted.

In the present invention, lixisenatide, as used herein, includes pharmaceutically acceptable salts thereof. The person skilled in the art knows suitable pharmaceutically acceptable salts of lixisenatide. An exemplary pharmaceutically acceptable salt of lixisenatide employed in the present invention is the acetate salt of lixisenatide.

In the present invention, insulin glargine, as used herein includes pharmaceutically acceptable salts thereof. The person skilled in the art knows suitable pharmaceutically acceptable salts of insulin glargine.

The formulation (a), as described herein, may be provided as a liquid composition, for example as an aqueous formulation.

The formulation (a), as described herein, can contain a preservative (e.g. phenol, m-cresol, p-cresol, a paraben), an isotonic agent (e.g. mannitol, sorbitol, lactose, dextrose, trehalose, sodium chloride, glycerol), buffer substances, salts, acids and alkalis and also further excipients. These substances can in each case be present individually or alternatively as mixtures.

The formulation (a), as described herein, may comprise a buffer substance. Buffer substances, such as, for example, phosphate, acetate, citrate, arginine, glycylglycine or TRIS (i.e. 2-amino-2-hydroxymethyl-1,3-propanediol) buffer and corresponding salts, can be present in a concentration of 5-250 mM, for example 10-100 mM. Further excipients can be, inter alia, salts or arginine.

The formulation (a), as described herein, may comprise a surfactant, for example, a non-ionic surfactant. For example, the surfactant can be a pharmaceutically customary surfactants such as, for example: partial and fatty acid esters and ethers of polyhydric alcohols such as of glycerol, sorbitol and the like (Span®, Tween®, for example Tween® 20 and Tween® 80, Myrj®, Brij®), Cremophor® or poloxamers. The surfactants are present in the pharmaceutical formulation (a) in a concentration of 5-200 μg/ml, for example of 5-120 μg/ml or 20-75 μg/ml.

The formulation (a), as described herein, may comprise a tonicity agent. A suitable tonicity agent may be selected from glycerol, dextrose, lactose, sorbitol, mannitol, glucose, NaCl, calcium or magnesium containing compounds such as CaCl₂. The concentration of glycerol, lactose, sorbitol, mannitol and glucose may be in the range of 100-250 mM. The concentration of NaCl may be up to 150 mM.

An exemplary tonicity agent is glycerol. Glycerol 85% can be present in an amount of 10-30 mg/mL, for example 20 mg/mL.

The pharmaceutical formulation (a), as described herein, may comprise methionine in a concentration selected from 0.3 mg/mL to 20 mg/mL, for example from 1 mg/ml to 5 mg/ml. An exemplary concentration of methionine is 3 mg/mL. For example, the liquid composition comprises L-methionine.

The pharmaceutical formulation (a), as described herein, may comprise a suitable preservative. A suitable preservative may be selected from phenol, m-cresol, benzyl alcohol and p-hydroxybenzoic acid ester. An exemplary preservative is m-cresol. The preservative, for example m-cresol, may be present in a concentration of up to 3 mg/mL, for example 1-3 mg/mL. For example, the concentration is 2.7 mg/mL.

The pharmaceutical formulation (a), as described herein, may comprise zinc. The zinc concentration may be in the range of 0-1000 μg/mL, for example 20-400 μg/mL zinc. An exemplary zinc concentration is 30 μg/mL. The zinc may be present in form of zinc chloride, but the salt is not limited to be zinc chloride.

The pH of the pharmaceutical formulation (a), as described herein, can be adjusted by hydrochloric acid or/and sodium hydroxide. The pH can be in the range of pH 1-6.8, pH 3.5-6.8, or pH 3.5-4.5. For example, the pH is in the range of 4.0-4.5. Exemplary pH values are 4.0 and 4.5.

In the present invention, the pharmaceutical formulation (a) may be administered to a type 2 diabetes mellitus patient in need thereof, in an amount sufficient to induce a therapeutic effect.

The pharmaceutical formulation (a), as described herein, may be administered parenterally. For example the pharmaceutical formulation (a), as described herein, may be administered by injection, such as, for example, by subcutaneous injection.

The pharmaceutical formulation (a) as described herein, may be an injectable formulation. Suitable injection devices, for instance the so-called “pens” comprising a cartridge comprising the formulation, and an injection needle, are known.

The formulation (a) may be administered by one injection per day. For example, the formulation (a) may be administered before breakfast, such as about 30 min before breakfast.

For example, 1 mL of the pharmaceutical formulation (a), comprising 50 μg/mL of lixisenatide, can contain:

| | | |
|---|---|---|
| Ingredient | Quantity | Standard |

Active ingredient

| Insulin glargine | 3.6378 | mg | Ph. Eur., USP |
|---|---|---|---|
| | [100 U] | | |
| Lixisenatide | 50 | μg | |

Excipients

| | | | |
|---|---|---|---|
| Glycerol (85%) | 20.0 | mg | Ph. Eur. |
| Methionine (L-methionine) | 3.0 | mg | Ph. Eur., USP |
| Metacresol (m-cresol) | 2.7 | mg | Ph. Eur., USP |
| Zinc chloride | 0.0626 | mg, | Ph. Eur., USP |

| | | |
|---|---|---|
| | corresponds to | |
| | 0.03 mg of zinc | |
| Hydrochloric acid | Up to pH = 4.5 | Ph. Eur., USP-NF |
| Sodium hydroxide | Up to pH = 4.5 | Ph. Eur., USP-NF |
| Water for injection | q.s. to 1.0 mL | Ph. Eur., USP |

This formulation is suitable for subcutaneous injection. In this formulation, the amount of zinc chloride reflects the total amount in the drug composition, including zinc (calculated as zinc chloride) from pharmaceutical substance insulin glargine and zinc chloride introduced during manufacture of the drug product.

For example, 1 mL of the pharmaceutical formulation (a), comprising 33 μg/mL of lixisenatide, can contain:

| | | |
|---|---|---|
| Ingredient | Quantity | Standard |

Active ingredient

| Insulin glargine | 3.6378 | mg | Ph. Eur., USP |
|---|---|---|---|
| | [100 U] | | |
| Lixisenatide | 33 | μg | |

Excipients

| | | | |
|---|---|---|---|
| Glycerol (85%) | 20.0 | mg | Ph. Eur. |
| Methionine (L-methionine) | 3.0 | mg | Ph. Eur., USP |
| Metacresol (m-cresol) | 2.7 | mg | Ph. Eur., USP |
| Zinc chloride | 0.0626 | mg, | Ph. Eur., USP |

| | | |
|---|---|---|
| | corresponds to | |
| | 0.03 mg of zinc | |
| Hydrochloric acid | Up to pH = 4.5 | Ph. Eur., USP-NF |
| Sodium hydroxide | Up to pH = 4.5 | Ph. Eur., USP-NF |
| Water for injection | q.s. to 1.0 mL | Ph. Eur., USP |

This formulation is suitable for subcutaneous injection. In this formulation, the amount of zinc chloride reflects the total amount in the drug composition, including zinc (calculated as zinc chloride) from pharmaceutical substance insulin glargine and zinc chloride introduced during manufacture of the drug product.

The pharmaceutical formulation (a), comprising 100 μg/mL of lixisenatide and 100 U/ml of insulin glargine, can contain the same excipients in the same concentrations of the formulations comprising 50 μg/mL of lixisenatide and 100 U/ml of insulin glargine, or 33 μg/mL of lixisenatide and 100 U/ml of insulin glargine.

Yet another aspect of the present invention relates to the use of a pharmaceutical combination, comprising
(a) a pharmaceutical formulation comprising
   (i) lixisenatide or/and a pharmaceutically acceptable salt thereof, and
   (ii) insulin glargine or/and a pharmaceutically acceptable salt thereof, and
(b) an SGLT2 inhibitor, or/and a pharmaceutically acceptable salt thereof, in the manufacture of a medicament for the treatment of type 2 diabetes mellitus. The patient may be any patient as described herein. The pharmaceutical formulation (a) may be any pharmaceutical formulation as described herein. The SGLT2 inhibitor (b) may be any SGLT2 inhibitor as described herein.

Yet another aspect of the present invention relates to a method of treatment of a type 2 diabetes mellitus patient in need thereof, the method comprising administering a pharmaceutical combination, comprising
(a) a pharmaceutical formulation comprising
   (i) lixisenatide or/and a pharmaceutically acceptable salt thereof, and
   (ii) insulin glargine or/and a pharmaceutically acceptable salt thereof, and
(b) an SGLT2 inhibitor, or/and a pharmaceutically acceptable salt thereof.

The patient may be any patient as described herein. The pharmaceutical formulation (a) may be any pharmaceutical formulation as described herein. The SGLT2 inhibitor (b) may be any SGLT2 inhibitor as described herein.

The following aspects are also subject of the present invention

Item 1. A pharmaceutical combination, comprising
(a) a pharmaceutical formulation comprising
   (i) lixisenatide or/and a pharmaceutically acceptable salt thereof, and
   (ii) insulin glargine or/and a pharmaceutically acceptable salt thereof, and
(b) an SGLT2 inhibitor, or/and a pharmaceutically acceptable salt thereof.

Item 2. The pharmaceutical combination according to item 1, further comprising (c) metformin or/and pharmaceutically acceptable salt thereof.

Item 3. The pharmaceutical combination according to item 1 or 2, wherein the SGLT2 inhibitor is selected from the group consisting of empagliflozin, canagliflozin, dapagliflozin and ertugliflozin.

Item 4. The pharmaceutical combination according to any one of the items 1 to 3, wherein the SGLT2 inhibitor is selected from the group consisting of empagliflozin, canagliflozin and dapagliflozin.

Item 5. The pharmaceutical combination according to any one of the preceding items, wherein the pharmaceutical formulation (a) comprises insulin glargine in a concentration of 100 to 500 U/mL.

Item 6. The pharmaceutical combination according to any one of the preceding items, wherein the pharmaceutical formulation (a) comprises insulin glargine in a concentration of 100 U/mL.

Item 7. The pharmaceutical combination according to any one of the preceding items, wherein the pharmaceutical formulation (a) comprises lixisenatide in a concentration of 20 to 150 μg/ml.

Item 8. The pharmaceutical combination according to any one of the preceding items, wherein the pharmaceutical formulation (a) comprises lixisenatide in a concentration of 33 μg/mL, 50 μg/mL or 100 μg/mL.

Item 9. The pharmaceutical combination according to any one of the items 1 to 8, wherein the pharmaceutical formulation comprises lixisenatide in a concentration of 33 μg/mL or 50 μg/mL.

Item 10. The pharmaceutical combination according to any one of the items 1 to 8, wherein the pharmaceutical formulation comprises lixisenatide in a concentration of 100 μg/mL.

Item 11. The pharmaceutical combination according to any one of the items 1 to 8, wherein the pharmaceutical formulation (a) comprises insulin glargine in a concentration of 100 U/ml, and lixisenatide in a concentration of 33 μg/mL, 50 μg/mL or 100 μg/mL.

Item 12. The pharmaceutical combination according to any one of the items 1 to 8 and 11, wherein the pharmaceutical formulation comprises insulin glargine in a concentration of 100 U/ml, and lixisenatide in a concentration of 33 μg/mL or 50 μg/mL.

Item 13. The pharmaceutical combination according to any one of the items 1 to 8 and 11, wherein the pharmaceutical formulation comprises insulin glargine in a concentration of 100 U/ml, and lixisenatide in a concentration of 100 μg/mL.

Item 14. The pharmaceutical combination according to any one of the preceding items, for use in the treatment of a type 2 diabetes mellitus patient.

Item 15. The pharmaceutical combination for use according to item 14, wherein the patient is a human patient.

Item 16. The pharmaceutical combination for use according to item 14 or 15, wherein the patient is an Asian patient.

Item 17. The pharmaceutical combination for use according to any one of the items 14 to 16, wherein the patient is a Chinese or a Japanese patient.

Item 18. The pharmaceutical combination for use according to item 14 or 15, wherein the patient is a Caucasian patient.

Item 19. The pharmaceutical combination for use according to any one of the items 14-18, wherein the type 2 diabetes mellitus to be treated is not adequately controlled with the SGLT2 inhibitor alone.

Item 20. The pharmaceutical combination for use according to any one of the items 14-18, wherein the type 2 diabetes mellitus to be treated is not adequately controlled with an SGLT2 inhibitor selected from empagliflozin, canagliflozin, dapagliflozin and ertugliflozin, or from the group consisting of empagliflozin, canagliflozin and dapagliflozin.

Item 21. The pharmaceutical combination for use according to any one of the items 14-18, wherein the type 2 diabetes mellitus to be treated is not adequately controlled with an SGLT2 inhibitor and an oral anti-diabetic alone.

Item 22. The pharmaceutical combination for use according to item 21, wherein the type 2 diabetes mellitus to be treated is not adequately controlled alone with
(a) an SGLT2 inhibitor selected from the group consisting of empagliflozin, canagliflozin, dapagliflozin and ertugliflozin, or from the group consisting of empagliflozin, canagliflozin and dapagliflozin, and
(b) an oral anti-diabetic selected from the group consisting of biguanides, thiazolidinediones, alpha-glucosidase inhibitors, glinides, and sulfonylureas, wherein the biguanide can be metformin.

Item 23. The pharmaceutical combination for use according to any one of the items 14-18, wherein the type 2 diabetes mellitus to be treated is not adequately controlled with the SGLT2 inhibitor and metformin alone, or with an SGLT2 inhibitor and a GLP-1 receptor agonist alone.

Item 24. The pharmaceutical combination for use according to item 23, wherein the type 2 diabetes mellitus to be treated is not adequately controlled alone with
(a) (i) an SGLT2 inhibitor selected from the group consisting of empagliflozin, canagliflozin, dapagliflozin and ertugliflozin, or from the group consisting of empagliflozin, canagliflozin and dapagliflozin, and (ii) metformin, or
(b) (i) an SGLT2 inhibitor selected from the group consisting of empagliflozin, canagliflozin, dapagliflozin and ertugliflozin, or from the group consisting of empagliflozin, canagliflozin and dapagliflozin, and (ii) a GLP-1 receptor agonist selected from the liraglutide, lixisenatide, exenatide, albiglutide and dulaglutide.

Item 25. The pharmaceutical combination for use according to any one of the items 14-18, wherein the type 2 diabetes mellitus to be treated is not adequately controlled with the SGLT2 inhibitor, metformin and a GLP-1 receptor agonist alone.

Item 26. The pharmaceutical combination for use according to item 25, wherein the type 2 diabetes mellitus to be treated is not adequately controlled alone with
(a) an SGLT2 inhibitor selected from the group consisting of empagliflozin, canagliflozin, dapagliflozin and ertugliflozin, or from the group consisting of empagliflozin, canagliflozin and dapagliflozin,
(b) an oral anti-diabetic selected from the group consisting of biguanides, thiazolidinediones, alpha-glucosidase inhibitors, glinides, and sulfonylureas, wherein the biguanide can be metformin, and
(c) GLP-1 receptor agonist selected from the liraglutide, lixisenatide, exenatide, albiglutide and dulaglutide.

Item 27. The pharmaceutical combination for use according to any one of the items 23 to 26, wherein the GLP-1 receptor agonist is selected from lixisenatide, exenatide, dulaglutide, and liraglutide.

Item 28. The pharmaceutical combination for use according to any one of the items 14-27, wherein the patient to be treated is obese.

Item 29. The pharmaceutical combination for use according to any one of the items 14-28, wherein the patient has a Body Mass Index (BMI) of at least 30 kg/m², at least 31 kg/m² or at least 32 kg/m².

Item 30. The pharmaceutical combination for use according to item 29, wherein the patient is a Caucasian patient.

Item 31. The pharmaceutical combination for use according to any one of the items 14 to 28, wherein the patient has a Body Mass Index (BMI) of at least 25 kg/m², or at least 26 kg/m².

Item 32. The pharmaceutical combination for use according to item 31, wherein the patient is an Asian patient, for example a Chinese or a Japanese patient.

Item 33. The pharmaceutical combination for use according to any one of the items 14 to 32, wherein prior to the onset of therapy with the combination according to any one of the items 1 to 8 the patient to be treated has a HbA1c of at least 7.5%.

Item 34. The pharmaceutical combination for use according to any one of the items 14 to 33, wherein prior to the onset of therapy with the combination according to any one of the items 1 to 8 the patient to be treated has a fasting plasma glucose concentration of at least mmol/L.

The invention is further illustrated by the following examples.

### Example 1

A 26-week randomized, open-label, active controlled, parallel-group, study assessing the efficacy and safety of the insulin glargine/lixisenatide fixed ratio combination in adults with Type 2 Diabetes inadequately controlled on GLP-1 receptor agonist and metformin (alone or with pioglitazone and/or SGLT2 inhibitors), followed by a fixed ratio combination single-arm 26-week extension period (EFC13794)

### Example 2

A randomized, 26-week, active-controlled, open-label, 2-treatment arm, parallel-group and multicenter study comparing the efficacy and safety of the insulin glargine/lixisenatide fixed-ratio combination (LixiLan) to lixisenatide on top of oral antidiabetic drugs in Japanese patients with type 2 diabetes mellitus Inadequately controlled on oral antidiabetic drugs with a 26-week safety extension period (EFC14112)

### Example 3

A randomized, 26-week, active-controlled, open label, 2-treatment arm, parallel-group and multicenter study comparing the efficacy and safety of the insulin glargine/lixisenatide fixed-ratio combination (LixiLan) to Insulin glargine on top of oral antidiabetic drugs in Japanese patients with type 2 diabetes mellitus inadequately controlled on oral antidiabetic drugs (EFC14114)

### Example 4

Concomitant Use of a Fixed-Dose Ratio Formulation of Insulin Glargine and Lixisenatide with SGLT2i—Subgroup Analysis of Example 1

The concomitant use of a fixed-dose ratio combination (FRC) of insulin glargine and lixisenatide with an SGLT2 inhibitor (SGLT2i) is supported by subgroup analyses of the patient group being subject of Example 1 (study EFC13794). In this study, 26 patients (10.1%) received concomitantly the FRC, metformin and a SGLT2i.

The FRC was provided in a single formulation, as described in Example 1.

An improvement was observed in efficacy results (change from baseline to Week 26 in glycated hemoglobin [HbA1c], fasting plasma glucose [FPG] and 2-hour postprandial plasma glucose [PPG]) in both treatment groups (Table 3, Table 4 and Table 5).

In patients receiving an SGLT2 inhibitor, the effect of the fixed-ratio formulation was larger than in the comparative treatment, compared with the patient group not receiving an SGLT2 inhibitor.

In patients receiving an SGLT2 inhibitor, after 26 weeks improvement in HbA1c was 0.88% in view of the active comparator (GLP-1 RA), compared with 0.61% in patients not receiving an SGLT2 inhibitor (Table 3).

In patients receiving an SGLT2 inhibitor, after 26 weeks improvement in fasting plasma glucose was 2.06 mmol/L in view of the active comparator (GLP-1 RA), compared with 1.64 mmol/L in patients not receiving an SGLT2 inhibitor (Table 4).

In patients receiving an SGLT2 inhibitor, after 26 weeks improvement in 2 hour postprandial glucose was 3.26 mmol/L in view of the active comparator (GLP-1 RA), compared with 2.81 mmol/L in patients not receiving an SGLT2 inhibitor (Table 5).

Overview of safety did not reveal relevant differences for SGLT2i users compared to non-users. In the FRC group, none of the SGLT2i users discontinued treatment due to an adverse event and no patients reported adverse events of body weight increase. Among the SGLT2i users no patients reported nausea or diarrhea in either treatment group, and only 1 patient reported vomiting in the FRC group (Table 6, Table 7).

Documented symptomatic hypoglycemia (plasma glucose 53.9 mmol/L [≤70 mg/dL]) was reported less frequently in the FRC group using SGLT2i versus non-users (0.72 events per patient year for SGLT2i users versus 1.62 for non-users) (Table 8).

TABLE 1
Demographics and patient characteristics at screening or baseline by
SGLT2 inhibitor use at screening-Randomized population (EFC13794)

| | SGLT2 inhibitor use at screening | | | | SGLT2 inhibitor use at screening | | | |
|---|---|---|---|---|---|---|---|---|
| | Yes | | | | No | | | |
| | (N = 52) | | | | (N = 462) | | | |
| | Fixed Ratio | | GLP-1 Receptor | | Fixed Ratio | | GLP-1 Receptor | |
| | Combination | | Agonist | | Combination | | Agonist | |
| | (N = 26) | | (N = 26) | | (N = 231) | | (N = 231) | |
| Age (years) | | | | | | | | |
| Number | 26 | | 26 | | 231 | | 231 | |
| Mean (SD) | 59.7 | (8.9) | 58.8 | (10.9) | 59.2 | (9.7) | 60.1 | (10.2) |
| Median | 59.5 | | 59.0 | | 59.0 | | 60.0 | |
| Min:Max | 40:82 | | 32:74 | | 30:78 | | 25:84 | |
| Age group (years) | | | | | | | | |
| [n (%)] | | | | | | | | |
| Number | 26 | | 26 | | 231 | | 231 | |
| <50 | 2 | (7.7%) | 3 | (11.5%) | 35 | (15.2%) | 37 | (16.0%) |
| ≥50-<65 | 17 | (65.4%) | 14 | (53.8%) | 113 | (48.9%) | 113 | (48.9%) |
| ≥65-<75 | 6 | (23.1%) | 9 | (34.6%) | 78 | (33.8%) | 66 | (28.6%) |
| ≥75 | 1 | (3.8%) | 0 | | 5 | (2.2%) | 15 | (6.5%) |
| Gender | | | | | | | | |
| [n (%)] | | | | | | | | |
| Number | 26 | | 26 | | 231 | | 231 | |
| Male | 11 | (42.3%) | 18 | (69.2%) | 115 | (49.8%) | 126 | (54.5%) |
| Female | 15 | (57.7%) | 8 | (30.8%) | 116 | (50.2%) | 105 | (45.5%) |
| Race | | | | | | | | |
| [n (%)] | | | | | | | | |
| Number | 26 | | 26 | | 231 | | 231 | |
| American Indian or Alaska | | | | | | | | |
| Native | 0 | | 0 | | 0 | | 0 | |
| Asian/Oriental | 0 | | 1 | (3.8%) | 3 | (1.3%) | 3 | (1.3%) |
| Black | 1 | (3.8%) | 0 | | 11 | (4.8%) | 7 | (3.0%) |
| Native Hawaiian or Other | | | | | | | | |
| Pacific Islander | 0 | | 0 | | 1 | (0.4%) | 0 | |
| White | 25 | (96.2%) | 24 | (92.3%) | 216 | (93.5%) | 220 | (95.2%) |
| Othera | 0 | | 1 | (3.8%) | 0 | | 1 | (0.4%) |
| Ethnicity | | | | | | | | |
| [n (%)] | | | | | | | | |
| Number | 26 | | 26 | | 231 | | 231 | |
| Hispanic or Latino | 4 | (15.4%) | 4 | (15.4%) | 23 | (10.0%) | 22 | (9.5%) |
| Not Hispanic or Latino | 22 | (84.6%) | 22 | (84.6%) | 208 | (90.0%) | 209 | (90.5%) |
| Unknown | 0 | | 0 | | 0 | | 0 | |
| HbA1c (%) at Visit 1 | | | | | | | | |
| (Week −2) | | | | | | | | |
| Number | 26 | | 26 | | 231 | | 231 | |
| Mean (SD) | 7.83 | (0.53) | 7.90 | (0.51) | 7.86 | (0.56) | 7.88 | (0.54) |
| Median | 7.90 | | 7.95 | | 7.80 | | 7.80 | |
| Min:Max | 7.0:9.0 | | 7.0:8.7 | | 7.0:9.0 | | 7.0:9.0 | |
| Randomization strata of Hba1c | | | | | | | | |
| (%) at Visit 1 (Week −2) [n (%)] | | | | | | | | |
| Number | 26 | | 26 | | 231 | | 231 | |
| <8 | 15 | (57.7%) | 13 | (50.0%) | 134 | (58.0%) | 134 | (58.0%) |
| ≥8 | 11 | (42.3%) | 13 | (50.0%) | 97 | (42.0%) | 97 | (42.0%) |
| Randomization strata of GLP-1 | | | | | | | | |
| receptor agonist subtype at | | | | | | | | |
| screening [n (%)] | | | | | | | | |
| Number | 26 | | 26 | | 231 | | 231 | |
| Once/twice daily formulation | 11 | (42.3%) | 11 | (42.3%) | 142 | (61.5%) | 143 | (61.9%) |
| Once weekly formulation | 15 | (57.7%) | 15 | (57.7%) | 89 | (38.5%) | 88 | (38.1%) |
| Baseline BMI | | | | | | | | |
| (kg/m²) | | | | | | | | |
| Number | 26 | | 26 | | 231 | | 231 | |
| Mean (SD) | 30.48 | (4.03) | 32.85 | (3.51) | 33.05 | (4.37) | 32.96 | (4.47) |
| Median | 30.75 | | 33.00 | | 33.00 | | 33.00 | |
| Min:Max | 24.5:39.0 | | 25.2:38.9 | | 21.2:40.0 | | 21.5:40.0 | |
| Baseline BMI categories | | | | | | | | |
| (kg/m²) | | | | | | | | |
| [n (%)] | | | | | | | | |
| Number | 26 | | 26 | | 231 | | 231 | |
| <30 | 12 | (46.2%) | 5 | (19.2%) | 59 | (25.5%) | 64 | (27.7%) |
| ≥30 | 14 | (53.8%) | 21 | (80.8%) | 172 | (74.5%) | 167 | (72.3%) |

BMI = Body Mass Index,
SGLT2 = Sodium glucose co-transporter 2
a Includes patients with more than one race, unknown or not reported

TABLE 2
Disease characteristics at screening or baseline by SGLT2 inhibitor use at screening -Randomized population (EFC13794)

| | SGLT2 inhibitor use at screening | | | | SGLT2 inhibitor use at screening | | | |
|---|---|---|---|---|---|---|---|---|
| | Yes | | | | No | | | |
| | (N = 52) | | | | (N = 462) | | | |
| | Fixed Ratio | | GLP-1 Receptor | | Fixed Ratio | | GLP-1 Receptor | |
| | Combination | | Agonist | | Combination | | Agonist | |
| | (N = 26) | | (N = 26) | | (N = 231) | | (N = 231) | |
| Duration of diabetes | | | | | | | | |
| (years) | | | | | | | | |
| Number | 26 | | 26 | | 231 | | 231 | |
| Mean (SD) | 12.31 | (6.86) | 10.91 | (5.39) | 11.11 | (7.49) | 10.96 | (6.16) |
| Median | 11.20 | | 9.87 | | 9.79 | | 9.83 | |
| Min:Max | 1.4:26.5 | | 2.5:21.2 | | 1.0:57.9 | | 1.4:40.1 | |
| Age at onset of Type 2 diabetes | | | | | | | | |
| (years) | | | | | | | | |
| Number | 26 | | 26 | | 231 | | 231 | |
| Mean (SD) | 47.3 | (9.3) | 47.8 | (9.9) | 48.0 | (10.0) | 49.1 | (9.1) |
| Median | 49.0 | | 51.0 | | 50.0 | | 50.0 | |
| Min:Max | 33:64 | | 27:63 | | 11:68 | | 19:69 | |
| Duration of GLP-1 receptor agonist | | | | | | | | |
| treatment (years) | | | | | | | | |
| Number | 26 | | 26 | | 231 | | 231 | |
| Mean (SD) | 1.75 | (1.24) | 2.06 | (1.70) | 1.91 | (1.81) | 1.90 | (1.87) |
| Median | 1.62 | | 1.41 | | 1.15 | | 1.06 | |
| Min:Max | 0.4:5.2 | | 0.5:6.4 | | 0.3:13.0 | | 0.3:12.1 | |
| GLP-1 receptor agonist | | | | | | | | |
| use by type at screening | | | | | | | | |
| in eCRF [n (%)] | | | | | | | | |
| Number | 26 | | 26 | | 231 | | 231 | |
| Once/twice daily formulation | 11 | (42.3%) | 11 | (42.3%) | 142 | (61.5%) | 143 | (61.9%) |
| Once weekly formulation | 15 | (57.7%) | 15 | (57.7%) | 89 | (38.5%) | 88 | (38.1%) |
| Daily dose of liraglutide | | | | | | | | |
| (mg) at baseline | | | | | | | | |
| Number | 11 | | 11 | | 124 | | 134 | |
| Mean (SD) | 1.69 | (0.24) | 1.69 | (0.24) | 1.66 | (0.26) | 1.66 | (0.29) |
| Median | 1.80 | | 1.80 | | 1.80 | | 1.80 | |
| Min:Max | 1.2:1.8 | | 1.2:1.8 | | 1.2:1.8 | | 1.2:3.0 | |
| Daily dose of exenatide | | | | | | | | |
| (μg) at baseline | | | | | | | | |
| Number | 0 | | 0 | | 18 | | 9 | |
| Mean (SD) | | | | | 18.33 | (3.83) | 17.78 | (4.41) |
| Median | | | | | 20.00 | | 20.00 | |
| Min:Max | | | | | 10.0:20.0 | | 10.0:20.0 | |
| Weekly dose of exenatide | | | | | | | | |
| extended-release | | | | | | | | |
| (mg) at baseline | | | | | | | | |
| Number | 4 | | 3 | | 41 | | 45 | |
| Mean (SD) | 2.00 | (0.00) | 2.00 | (0.00) | 2.00 | (0.00) | 2.00 | (0.00) |
| Median | 2.00 | | 2.00 | | 2.00 | | 2.00 | |
| Min:Max | 2.0:2.0 | | 2.0:2.0 | | 2.0:2.0 | | 2.0:2.0 | |
| Weekly dose of albiglutide | | | | | | | | |
| (mg) at baseline | | | | | | | | |
| Number | 0 | | 2 | | 5 | | 2 | |
| Mean (SD) | | | 40.00 | (14.14) | 50.00 | (0.00) | 50.00 | (0.00) |
| Median | | | 40.00 | | 50.00 | | 50.00 | |
| Min:Max | | | 30.0:50.0 | | 50.0:50.0 | | 50.0:50.0 | |
| Weekly dose of dulaglutide | | | | | | | | |
| (mg) at baseline | | | | | | | | |
| Number | 11 | | 10 | | 43 | | 41 | |
| Mean (SD) | 1.36 | (0.30) | 1.43 | (0.24) | 1.45 | (0.19) | 1.39 | (0.27) |
| Median | 1.50 | | 1.50 | | 1.50 | | 1.50 | |
| Min:Max | 0.8:1.5 | | 0.8:1.5 | | 0.8:1.5 | | 0.8:1.5 | |
| Pioglitazone use at screening | | | | | | | | |
| recorded in eCRF [n (%)] | | | | | | | | |
| Number | 26 | | 26 | | 231 | | 231 | |
| Yes | 1 | (3.8%) | 1 | (3.8%) | 11 | (4.8%) | 21 | (9.1%) |
| No | 25 | (96.2%) | 25 | (96.2%) | 220 | (95.2%) | 210 | (90.9%) |
| Daily dose of pioglitazone | | | | | | | | |
| at baseline (mg) | | | | | | | | |
| Number | 1 | | 1 | | 11 | | 21 | |
| Mean (SD) | 30.00 | (NC) | 30.00 | (NC) | 31.36 | (10.51) | 32.86 | (9.02) |
| Median | 30.00 | | 30.00 | | 30.00 | | 30.00 | |
| Min:Max | 30.0:30.0 | | 30.0:30.0 | | 15.0:45.0 | | 15.0:45.0 | |
| SGLT2 inhibitor use at screening | | | | | | | | |
| [n (%)] | | | | | | | | |
| Number | 26 | | 26 | | 231 | | 231 | |
| Yes | 26 | (100%) | 26 | (100%) | 0 | | 0 | |
| No | 0 | | 0 | | 231 | (100%) | 231 | (100%) |
| Daily dose of SGLT2 inhibitor | | | | | | | | |
| at baseline (mg) | | | | | | | | |
| Canagliflozin | | | | | | | | |
| Number | 7 | | 12 | | 0 | | 0 | |
| Mean (SD) | 214.29 | (106.90) | 283.33 | (57.74) | | | | |
| Median | 300.00 | | 300.00 | | | | | |
| Min:Max | 100.0:300.0 | | 100.0:300.0 | | | | | |
| Empagliflozin | | | | | | | | |
| Number | 6 | | 9 | | 0 | | 0 | |
| Mean (SD) | 15.42 | (7.49) | 16.67 | (8.20) | | | | |
| Median | 11.25 | | 12.50 | | | | | |
| Min:Max | 10.0:25.0 | | 5.0:25.0 | | | | | |
| Dapagliflozin | | | | | | | | |
| Number | 13 | | 5 | | 0 | | 0 | |
| Mean (SD) | 9.62 | (3.80) | 9.00 | (2.24) | | | | |
| Median | 10.00 | | 10.00 | | | | | |
| Min:Max | 5.0:20.0 | | 5.0:10.0 | | | | | |
| Duration of metformin treatment | | | | | | | | |
| (years) | | | | | | | | |
| Number | 26 | | 26 | | 231 | | 231 | |
| Mean (SD) | 7.35 | (6.34) | 8.59 | (5.35) | 7.18 | (5.17) | 8.06 | (5.20) |
| Median | 4.21 | | 7.16 | | 6.56 | | 7.20 | |
| Min:Max | 0.5:18.6 | | 0.7:21.2 | | 0.3:28.8 | | 0.4:29.4 | |
| Daily dose of metformin | | | | | | | | |
| at baseline (mg) | | | | | | | | |
| Number | 26 | | 26 | | 231 | | 231 | |
| Mean (SD) | 1730.77 | (496.00) | 1805.77 | (529.21) | 1993.51 | (420.04) | 2056.06 | (488.15) |
| Median | 2000.00 | | 2000.00 | | 2000.00 | | 2000.00 | |
| Min:Max | 1000.0:2550.0 | | 500.0:3000.0 | | 500.0:3000.0 | | 500.0:3000.0 | |
| Categorized daily dose | | | | | | | | |
| of metformin at baseline | | | | | | | | |
| (mg) [n (%)] | | | | | | | | |
| Number | 26 | | 26 | | 231 | | 231 | |
| <1500 mg | 7 | (26.9%) | 5 | (19.2%) | 11 | (4.8%) | 13 | (5.6%) |
| ≥1500-<2500 mg | 17 | (65.4%) | 19 | (73.1%) | 183 | (79.2%) | 162 | (70.1%) |
| ≥2500-<3000 mg | 2 | (7.7%) | 1 | (3.8%) | 25 | (10.8%) | 33 | (14.3%) |
| ≥3000 mg | 0 | | 1 | (3.8%) | 12 | (5.2%) | 23 | (10.0%) |
| Diabetic retinopathy | | | | | | | | |
| [n (%)] | | | | | | | | |
| Number | 26 | | 26 | | 231 | | 231 | |
| Yes | 4 | (15.4%) | 1 | (3.8%) | 16 | (6.9%) | 13 | (5.6%) |
| No | 22 | (84.6%) | 25 | (96.2%) | 215 | (93.1%) | 218 | (94.4%) |
| Diabetic proliferative retinopathy | | | | | | | | |
| [n (%)] | | | | | | | | |
| Number | 26 | | 26 | | 231 | | 231 | |
| Yes | 0 | | 0 | | 2 | (0.9%) | 2 | (0.9%) |
| No | 26 | (100%) | 26 | (100%) | 229 | (99.1%) | 229 | (99.1%) |
| Diabetic neuropathy | | | | | | | | |
| [n (%)] | | | | | | | | |
| Number | 26 | | 26 | | 231 | | 231 | |
| Yes | 8 | (30.8%) | 7 | (26.9%) | 54 | (23.4%) | 52 | (22.5%) |
| No | 18 | (69.2%) | 19 | (73.1%) | 177 | (76.6%) | 179 | (77.5%) |
| Diabetic nephropathy | | | | | | | | |
| [n (%)] | | | | | | | | |
| Number | 26 | | 26 | | 231 | | 231 | |
| Yes | 1 | (3.8%) | 4 | (15.4%) | 19 | (8.2%) | 22 | (9.5%) |
| No | 25 | (96.2%) | 22 | (84.6%) | 212 | (91.8%) | 209 | (90.5%) |
| Baseline urinary albumin/creatinine | | | | | | | | |
| ratio (mg/g) | | | | | | | | |
| [n (%)] | | | | | | | | |
| Number | 26 | | 26 | | 227 | | 230 | |
| <30 (normal) | 21 | (80.8%) | 23 | (88.5%) | 176 | (77.5%) | 180 | (78.3%) |
| ≥30 to <300 (microalbuminuria) | 5 | (19.2%) | 3 | (11.5%) | 43 | (18.9%) | 42 | (18.3%) |
| ≥300 (macroalbuminuria) | 0 | | 0 | | 8 | (3.5%) | 8 | (3.5%) |
| Estimated eGFR at screening | | | | | | | | |
| (mL/min/1.73 m²) | | | | | | | | |
| Number | 26 | | 26 | | 231 | | 231 | |
| Mean (SD) | 93.51 | (27.52) | 82.70 | (18.57) | 88.45 | (24.57) | 86.05 | (23.02) |
| Median | 92.19 | | 84.43 | | 85.36 | | 84.06 | |
| Min:Max | 46.5:169.0 | | 42.5:112.5 | | 40.9:296.4 | | 39.5:171.8 | |
| Estimated eGFR at screening | | | | | | | | |
| (mL/min/1.73 m²) | | | | | | | | |
| [n (%)] | | | | | | | | |
| Number | 26 | | 26 | | 231 | | 231 | |
| <15 (End stage renal disease) | 0 | | 0 | | 0 | | 0 | |
| ≥15-<30 (Severe decrease in GFR) | 0 | | 0 | | 0 | | 0 | |
| ≥30-<60 (Moderate decrease in GFR) | 3 | (11.5%) | 4 | (15.4%) | 14 | (6.1%) | 27 | (11.7%) |
| ≥60-<90 (Mild decrease in GFR) | 10 | (38.5%) | 13 | (50.0%) | 122 | (52.8%) | 113 | (48.9%) |
| ≥90 (Normal) | 13 | (50.0%) | 9 | (34.6%) | 95 | (41.1%) | 91 | (39.4%) |

SGLT2 = Sodium glucose co-transporter 2,
eGFR = estimated Glomerular Filtration Rate.
Duration of diabetes (years) = (Date of informed consent − Date of diagnosis of diabetes + 1)/365.25
Age at onset of Type 2 diabetes (years) = (year of diagnosis of diabetes − year of birth)
eGFR is derived using the 4 variable Modification of Diet in Renal Disease formula.
Urine albumin/creatinine ratio is presented in mg/g, and the conversion factor to the standard international unit mg/mmoL is 0.1130.

TABLE 3
Mean change in HbA1c (%) from baseline to Week 26 by SGLT2 inhibitor use at
screening-mITT population (EFC13794)

| | Fixed Ratio Combination | | | | | |
|---|---|---|---|---|---|---|
| | (N = 252) | | | | | |
| | | | Compared to GLP-1 Receptor | | | |
| | | | Agonist | | GLP-1 Receptor Agonist | |
| | | | Difference in | | (N = 253) | |
| | N | LSMean (SE)a | LSMean (SE)a | 95% CIa | N | LSMean (SE)a |
| SGLT2 inhibitor | | | | | | |
| use at screening | | | | | | |
| Yes | 22 | −1.15 (0.161) | −0.88 (0.217) | (−1.306, −0.455) | 26 | −0.27 (0.148) |
| No | 228 | −1.01 (0.051) | −0.61 (0.070) | (−0.751, −0.475) | 227 | −0.40 (0.051) |

SGLT2 = Sodium glucose co-transporter 2.
aMixed-effect model with repeated measures (MMRM) with treatment groups (fixed ratio combination and GLP-1 receptor agonist), randomization strata of HbA1c (<8.0%, ≥8.0%) at Visit 1 (Week-2), randomization strata of GLP-1 receptor agonist subtype (once/twice daily formulations, once weekly formulations) at screening, scheduled visit, subgroup factor, treatment-by-vist, treatment-by-subgroup factor, visit-by-subgroup factor, treatment-by-visit-by-subgroup factor, and world region as fixed effects, and baseline HbA1c value-by-visit interaction as a covariate.
Difference in LSMean between fixed ratio combination and GLP-1 receptor agonist.
The analysis includes all scheduled measurements obtained during the 26-week randomized treatment period, including those obtained after IMP discontinuation or introduction of rescue therapy.
Included are patients who have measurements at baseline and post-baseline.
LS Mean and difference in LS Mean were provided for categories with ≥5 patients in each treatment group.

TABLE 4
Mean change in fasting plasma glucose (mmol/L) from baseline to Week 26 by SGLT2
inhibitor use at screening-mITT population (EFC13794)

| | | Fixed Ratio Combination | | | | |
|---|---|---|---|---|---|---|
| | | (N = 252) | | | | |
| | | | Compared to GLP-1 Receptor | | | |
| | | | Agonist | | GLP-1 Receptor Agonist | |
| SGLT2 inhibitor | | | Difference in | | (N = 253) | |
| use at screening | N | LSMean (SE)a | LSMean (SE)a | 95% CIa | N | LSMean (SE)a |
| Yes | 23 | −2.83 (0.394) | −2.06 (0.536) | (−3.114, −1.009) | 26 | −0.77 (0.369) |
| No | 228 | −2.22 (0.126) | −1.64 (0.177) | (−1.984, −1.288) | 227 | −0.58 (0.127) |

SGLT2 = Sodium glucose co-transporter 2.
aMixed-effect model with repeated measures (MMRM) with treatment groups (fixed ratio combination and GLP-1 receptor agonist), randomization strata of HbA1c (<8.0%, ≥8.0%) at Visit 1 (Week-2), randomization strata of GLP-1 receptor agonist subtype (once/twice daily formulations, once weekly formulations) at screening, scheduled visit, subgroup factor, treatment-by-visit, treatment-by-subgroup factor, visit-by-subgroup factor, treatment-by-visit-by-subgroup factor, and world region as fixed effects, and baseline fasting plasma glucose value-by-visit interaction as a covariate.
Difference in LSMean between fixed ratio combination and GLP-1 receptor agonist.
The analysis includes all scheduled measurements obtained during the 26-week randomized treatment period, including those obtained after IMP discontinuation or introduction of rescue therapy.
Included are patients who have measurements at baseline and post-baseline.
LS Mean and difference in LS Mean were provided for categories with ≥5 patients in each treatment group.

TABLE 5
Mean change in 2-hour postprandial plasma glucose (mmol/L) during a standardized meal
test from baseline to Week 26 by SGLT2 inhibitor use at screening-mITT population (EFC13794)

| | Fixed Ratio Combination | | | | | |
|---|---|---|---|---|---|---|
| | (N = 252) | | | | | |
| | | | Compared to GLP-1 Receptor | | | |
| | | | Agonist | | GLP-1 Receptor Agonist | |
| SGLT2 inhibitor | | | Difference in | | (N = 253) | |
| use at screening | N | LSMean (SE)a | LSMean (SE)a | 95% CIa | N | LSMean (SE)a |
| Yes | 21 | −5.04 (0.665) | −3.26 (0.896) | (−5.021, −1.498) | 25 | −1.78 (0.619) |
| No | 194 | −3.83 (0.223) | −2.81 (0.306) | (−3.411, −2.209) | 197 | −1.02 (0.219) |

SGLT2 = Sodium glucose co-transporter 2.
aAnalysis of covariance (ANCOVA) model with treatment groups (fixed ratio combination and GLP-1 receptor agonist), randomization strata of HbA1c (<8.0%, ≥8.0%) at Visit 1 (Week-2), randomization strata of GLP-1 receptor agonist subtype (once/twice daily formulations, once weekly formulations) at screening, subgroup factor, treatment-by-subgroup factor and world region as fixed effects and baseline 2-hour postprandial plasma glucose value as a covariate.
Difference in LSMean between fixed ratio combination and GLP-1 receptor agonist.
The analysis includes all scheduled measurements obtained during the 26-week randomized treatment period, including those obtained after IMP discontinuation or introduction of rescue therapy.
Patients with both baseline and Week 26 (LOCF) measurements were included.
LS Mean and difference in LS Mean were provided for categories with ≥5 patients in each treatment group.

TABLE 6
Overview of adverse event profile: treatment-emergent adverse events by SGLT-2 inhibitor use
at screening during the 26-week randomized treatment period-Safety population (EFC13794)

| | SGLT2 inhibitor use at screening | | SGLT2 inhibitor use at screening | |
|---|---|---|---|---|
| | Yes | | No | |
| | (N = 51) | | (N = 460) | |
| | Fixed Ratio | GLP-1 Receptor | Fixed Ratio | GLP-1 Receptor |
| | Combination | Agonist | Combination | Agonist |
| n (%) | (N = 25) | (N = 26) | (N = 230) | (N = 230) |
| Patients with any TEAE | 14 (56.0%) | 15 (57.7%) | 149 (64.8%) | 106 (46.1%) |
| Patients with any serious TEAE | 2 (8.0%) | 1 (3.8%) | 8 (3.5%) | 8 (3.5%) |
| Patients with any TEAE leading | 0 | 0 | 0 | 0 |
| to death | | | | |
| Patients with any TEAE leading | 0 | 0 | 9 (3.9%) | 0 |
| to permanent treatment | | | | |
| discontinuation | | | | |

SGLT2 = Sodium glucose co-transporter 2
TEAE: Treatment-emergent adverse event
n (%) = number and percentage of patients with at least one TEAE

TABLE 7
Number (%) of patients experiencing common TEAE(s) (PT ≥2% in any treatment
group) by primary SOC and PT and by SGLT-2 inhibitor use at screening
during the 26-week on-treatment period-Safety population (EFC13794)

| | SGLT2 inhibitor use at screening | | | | SGLT2 inhibitor use at screening | | | |
|---|---|---|---|---|---|---|---|---|
| | Yes | | | | No | | | |
| Primary System | (N = 51) | | | | (N = 460) | | | |
| Organ Class | Fixed Ratio | | GLP-1 Receptor | | Fixed Ratio | | GLP-1 Receptor | |
| Preferred Term | Combination | | Agonist | | Combination | | Agonist | |
| [n (%)] | (N = 25) | | (N = 26) | | (N = 230) | | (N = 230) | |
| Any TEAE | 14 | (56.0%) | 15 | (57.7%) | 149 | (64.8%) | 106 | (46.1%) |
| Infections and infestations | 5 | (20.0%) | 10 | (38.5%) | 73 | (31.7%) | 58 | (25.2%) |
| Nasopharyngitis | 3 | (12.0%) | 5 | (19.2%) | 22 | (9.6%) | 18 | (7.8%) |
| Influenza | 1 | (4.0%) | 2 | (7.7%) | 10 | (4.3%) | 4 | (1.7%) |
| Upper respiratory tract | 0 | | 2 | (7.7%) | 9 | (3.9%) | 10 | (4.3%) |
| infection | | | | | | | | |
| Urinary tract infection | 0 | | 0 | | 7 | (3.0%) | 3 | (1.3%) |
| Bronchitis | 0 | | 0 | | 6 | (2.6%) | 5 | (2.2%) |
| Pneumonia | 0 | | 0 | | 6 | (2.6%) | 3 | (1.3%) |
| Sinusitis | 2 | (8.0%) | 0 | | 4 | (1.7%) | 7 | (3.0%) |
| Metabolism and nutrition | 0 | | 0 | | 13 | (5.7%) | 1 | (0.4%) |
| disorders | | | | | | | | |
| Increased appetite | 0 | | 0 | | 6 | (2.6%) | 0 | |
| Nervous system disorders | 3 | (12.0%) | 0 | | 27 | (11.7%) | 13 | (5.7%) |
| Headache | 0 | | 0 | | 10 | (4.3%) | 6 | (2.6%) |
| Dizziness | 0 | | 0 | | 6 | (2.6%) | 2 | (0.9%) |
| Gastrointestinal disorders | 1 | (4.0%) | 3 | (11.5%) | 54 | (23.5%) | 23 | (10.0%) |
| Nausea | 0 | | 0 | | 22 | (9.6%) | 6 | (2.6%) |
| Diarrhoea | 0 | | 0 | | 14 | (6.1%) | 6 | (2.6%) |
| Vomiting | 1 | (4.0%) | 0 | | 7 | (3.0%) | 2 | (0.9%) |
| Toothache | 0 | | 0 | | 6 | (2.6%) | 3 | (1.3%) |
| Musculoskeletal and | 2 | (8.0%) | 3 | (11.5%) | 21 | (9.1%) | 15 | (6.5%) |
| connective tissue disorders | | | | | | | | |

| | | | | | | | |
|---|---|---|---|---|---|---|---|
| Back pain | 0 | 1 | (3.8%) | 4 | (1.7%) | 6 | (2.6%) |
| Investigations | 0 | 1 | (3.8%) | 15 | (6.5%) | 3 | (1.3%) |

| | | | | | |
|---|---|---|---|---|---|
| Weight increased | 0 | 0 | 7 | (3.0%) | 0 |

TEAE: Treatment-emergent adverse event,
SOC: System Organ Class,
PT: Preferred Term,
SGLT2 = Sodium glucose co-transporter 2.
MedDRA version: 21.0
n (%) = number and percentage of patients with at least one TEAE.
Note:
Table sorted by SOC internationally agreed order and PT sorted by decreasing frequency within a SOC according to all TEAE summary.
Only SOC with at least one PT ≥2% in at least one treatment group are presented.

TABLE 8
Summary of documented symptomatic hypoglycemia recorded on the dedicated
eCRF page and meeting protocol definition by SGLT2 inhibitor use at screening
during the 26-week on-treatment period-Safety population (EFC13794)

| | SGLT2 inhibitor use at screening | | SGLT2 inhibitor use at screening | |
|---|---|---|---|---|
| | Yes | | No | |
| | (N = 51) | | (N = 460) | |
| | Fixed Ratio | GLP-1 Receptor | Fixed Ratio | GLP-1 Receptor |
| | Combination | Agonist | Combination | Agonist |
| Type | (N = 25) | (N = 26) | (N = 230) | (N = 230) |
| Total patient years of exposure | 11.0 | 13.2 | 110.3 | 113.8 |
| Documented symptomatic | | | | |
| hypoglycemia (plasma | | | | |
| glucose ≤70 mg/dL | | | | |
| [3.9 mmol/L]) | | | | |
| Number of patients with events, | 6 (24.0%) | 0 | 65 (28.3%) | 6 (2.6%) |
| n (%) | | | | |
| Number of patients with events | 0.54 | 0 | 0.59 | 0.05 |
| per patient yeara | | | | |
| Number of events | 8 | 0 | 179 | 10 |
| Number of events per patient | 0.72 | 0 | 1.62 | 0.09 |
| yearb | | | | |

SGLT2 = Sodium glucose co-transporter 2
aCalculated as number of patients with events divided by total exposure + 1 day in patient years for daily formulation and number of patients with events divided by total exposure + 7 days in patient years for weekly formulation.
bCalculated as number of events divided by total exposure + 1 day in patient years for daily formulation and number of events divided by total exposure + 7 day in patient years for weekly formulation.
Documented symptomatic hypoglycemia = symptomatic hypoglycemia recorded on the dedicated eCRF and meeting protocol definition for documented symptomatic hypoglycemia.
The 26-week on-treatment period is defined as the time from the first injection of investigational medicinal product (IMP) up to 1 day after the last injection of daily IMP (7 days after the last injection of weekly IMP) for patients not eligible to enter the extension period or Visit 28/Week 26 (or Day 183 if Visit 28/Week 26 is missing) for patients eligible to enter the extension period, regardless of the introduction of rescue therapy.

### Example 5

Concomitant Use of a Fixed-Dose Ratio Formulation of Insulin Glargine and Lixisenatide with SGLT2i—Subgroup Analysis of Example 2

The concomitant use of a fixed-dose ratio combination (FRC) of insulin glargine and lixisenatide with an SGLT2 inhibitor (SGLT2i) is supported by subgroup analyses of the patient group being subject of Example 2 (study EFC14112). In this study, 34 patients (21.1%) received concomitantly the FRC and a SGLT2i.

The FRC was provided in a single formulation, as described in Example 2.

Efficacy results (change from baseline to Week 26 in HbA1c and FPG) in both treatment groups were generally similar in SGLT2i users and non-users (Table 3, Table 4).

Overview of safety did not reveal differences between SGLT2i users and non-users (Table 5). With regard to common treatment-emergent adverse events (TEAEs) (Table 6), TEAEs in the gastrointestinal disorder System Organ Class (SOC) were reported less frequently in the FRC treatment group in SGLT2i users when compared to the SGLT2i non-users (17.6% versus 32.3%, respectively). Similarly, documented symptomatic hypoglycemia (plasma glucose ≤3.9 mmol/L [≤70 mg/dL]) in the FRC group was reported less frequently in SGLT2i users compared to non-users (number of events per patient year: 0.18 and 1.14, respectively) (Table 7). No new safety signal was identified when patients in the FRC treatment group were using SGLT2i as background therapy compared to SGLT2i non-users.

TABLE 1
Demographics and patient characteristics at screening or baseline by
SGLT2 inhibitor use at screening-Randomized population (EFC14112)

| | SGLT2 inhibitor use at screening | | | | SGLT2 inhibitor use at screening | | | |
|---|---|---|---|---|---|---|---|---|
| | Yes | | | | No | | | |
| | (N = 64) | | | | (N = 257) | | | |
| | Fixed Ratio | | | | Fixed Ratio | | | |
| | Combination | | Lixisenatide | | Combination | | Lixisenatide | |
| | (N = 34) | | (N = 30) | | (N = 127) | | (N = 130) | |
| Age (years) | | | | | | | | |
| Number | 34 | | 30 | | 127 | | 130 | |
| Mean (SD) | 52.4 | (10.7) | 50.2 | (10.6) | 59.9 | (9.1) | 59.5 | (11.0) |
| Median | 52.0 | | 51.0 | | 61.0 | | 60.0 | |
| Min:Max | 26:72 | | 27:72 | | 37:81 | | 33:82 | |
| Age group (years) | | | | | | | | |
| [n (%)] | | | | | | | | |
| Number | 34 | | 30 | | 127 | | 130 | |
| <65 | 28 | (82.4%) | 27 | (90.0%) | 87 | (68.5%) | 83 | (63.8%) |
| ≥65 to <75 | 6 | (17.6%) | 3 | (10.0%) | 35 | (27.6%) | 36 | (27.7%) |
| ≥75 | 0 | | 0 | | 5 | (3.9%) | 11 | (8.5%) |
| Gender [n (%)] | | | | | | | | |
| Number | 34 | | 30 | | 127 | | 130 | |
| Male | 20 | (58.8%) | 19 | (63.3%) | 84 | (66.1%) | 83 | (63.8%) |
| Female | 14 | (41.2%) | 11 | (36.7%) | 43 | (33.9%) | 47 | (36.2%) |
| Race [n (%)] | | | | | | | | |
| Number | 34 | | 30 | | 127 | | 130 | |
| Asian | 34 | (100%) | 30 | (100%) | 127 | (100%) | 130 | (100%) |
| HbA1c (%) at screening | | | | | | | | |
| Number | 34 | | 30 | | 127 | | 130 | |
| Mean (SD) | 8.47 | (0.65) | 8.35 | (0.64) | 8.42 | (0.62) | 8.44 | (0.64) |
| Median | 8.50 | | 8.10 | | 8.30 | | 8.30 | |
| Min:Max | 7.6:9.8 | | 7.5:9.7 | | 7.5:10.0 | | 7.5:10.0 | |
| HbA1c (%) categories | | | | | | | | |
| at screening | | | | | | | | |
| [n (%)] | | | | | | | | |
| Number | 34 | | 30 | | 127 | | 130 | |
| <8 | 9 | (26.5%) | 12 | (40.0%) | 34 | (26.8%) | 31 | (23.8%) |
| ≥8 | 25 | (73.5%) | 18 | (60.0%) | 93 | (73.2%) | 99 | (76.2%) |
| Randomization strata | | | | | | | | |
| of HbA1c (%) from IVRS/IWRS | | | | | | | | |
| Number | 34 | | 30 | | 127 | | 130 | |
| <8 | 9 | (26.5%) | 12 | (40.0%) | 34 | (26.8%) | 31 | (23.8%) |
| ≥8 | 25 | (73.5%) | 18 | (60.0%) | 93 | (73.2%) | 99 | (76.2%) |
| Randomization strata of DPP-4 | | | | | | | | |
| inhibitor use at screening from | | | | | | | | |
| IVRS/IWRS | | | | | | | | |
| Number | 34 | | 30 | | 127 | | 130 | |
| Yes | 6 | (17.6%) | 3 | (10.0%) | 66 | (52.0%) | 68 | (52.3%) |
| No | 28 | (82.4%) | 27 | (90.0%) | 61 | (48.0%) | 62 | (47.7%) |
| Baseline BMI | | | | | | | | |
| (kg/m²) | | | | | | | | |
| Number | 34 | | 30 | | 127 | | 130 | |
| Mean (SD) | 28.34 | (5.15) | 27.36 | (4.59) | 26.38 | (4.16) | 26.73 | (4.08) |
| Median | 27.36 | | 27.56 | | 26.40 | | 26.32 | |
| Min:Max | 20.5:39.6 | | 19.4:38.7 | | 18.8:40.2 | | 18.9:39.4 | |
| Baseline BMI categories | | | | | | | | |
| (kg/m²) | | | | | | | | |
| [n (%)] | | | | | | | | |
| Number | 34 | | 30 | | 127 | | 130 | |
| <25 | 10 | (29.4%) | 8 | (26.7%) | 52 | (40.9%) | 44 | (33.8%) |
| ≥25 to <30 | 14 | (41.2%) | 14 | (46.7%) | 53 | (41.7%) | 62 | (47.7%) |
| ≥30 | 10 | (29.4%) | 8 | (26.7%) | 22 | (17.3%) | 24 | (18.5%) |
| FPG (mmol/L) at screening | | | | | | | | |
| Number | 34 | | 30 | | 127 | | 130 | |
| Mean (SD) | 9.47 | (1.54) | 9.01 | (1.57) | 10.00 | (1.51) | 10.03 | (1.70) |
| Median | 9.52 | | 8.77 | | 9.77 | | 9.94 | |
| Min:Max | 6.5:12.1 | | 6.7:11.9 | | 6.1:13.4 | | 6.5:13.5 | |
| FPG (mg/dL) at screening | | | | | | | | |
| Number | 34 | | 30 | | 127 | | 130 | |
| Mean (SD) | 170.7 | (27.8) | 162.3 | (28.3) | 180.2 | (27.2) | 180.7 | (30.6) |

| | | | | |
|---|---|---|---|---|
| Median | 171.5 | 158.0 | 176.0 | 179.0 |
| Min:Max | 117:218 | 121:215 | 109:241 | 117:244 |

SGLT2 = Sodium glucose co-transporter 2,
BMI = Body Mass Index,
FPG = Fasting plasma glucose,
DPP-4 = Dipeptidyl-peptidase-4,
IVRS/IWRS = Interactive Voice/Web Response System.

TABLE 2
Disease characteristics at screening or baseline by SGLT2 inhibitor
use at screening-Randomized population (EFC14112)

| | SGLT2 inhibitor use at screening | | | | SGLT2 inhibitor use at screening | | | |
|---|---|---|---|---|---|---|---|---|
| | Yes | | | | No | | | |
| | (N = 64) | | | | (N = 257) | | | |
| | Fixed Ratio | | | | Fixed Ratio | | | |
| | Combination | | Lixisenatide | | Combination | | Lixisenatide | |
| | (N = 34) | | (N = 30) | | (N = 127) | | (N = 130) | |
| Duration of diabetes | | | | | | | | |
| (years) | | | | | | | | |
| Number | 34 | | 30 | | 127 | | 130 | |
| Mean (SD) | 5.21 | (3.83) | 5.73 | (5.52) | 8.90 | (6.30) | 10.03 | (6.32) |
| Median | 3.88 | | 4.56 | | 7.90 | | 9.35 | |
| Min:Max | 1.1:16.8 | | 1.1:29.4 | | 1.0:38.5 | | 1.0:30.4 | |
| Age at onset of Type 2 diabetes | | | | | | | | |
| (years) | | | | | | | | |
| Number | 34 | | 30 | | 127 | | 130 | |
| Mean (SD) | 47.1 | (10.1) | 44.5 | (9.8) | 51.0 | (9.9) | 49.4 | (10.2) |
| Median | 47.0 | | 44.0 | | 52.0 | | 50.0 | |
| Min:Max | 25:69 | | 26:61 | | 24:77 | | 24:79 | |
| Prior use of insulin | | | | | | | | |
| [n (%)] | | | | | | | | |
| Number | 34 | | 30 | | 127 | | 130 | |
| Yes | 3 | (8.8%) | 1 | (3.3%) | 8 | (6.3%) | 10 | (7.7%) |
| No | 31 | (91.2%) | 29 | (96.7%) | 119 | (93.7%) | 120 | (92.3%) |
| Prior use of GLP-1 receptor agonist | | | | | | | | |
| [n (%)] | | | | | | | | |
| Number | 34 | | 30 | | 127 | | 130 | |
| Yes | 10 | (29.4%) | 7 | (23.3%) | 24 | (18.9%) | 26 | (20.0%) |
| No | 24 | (70.6%) | 23 | (76.7%) | 103 | (81.1%) | 104 | (80.0%) |
| History of gestational diabetes | | | | | | | | |
| [n (%)] | | | | | | | | |
| Number (Female) | 14 | | 11 | | 43 | | 47 | |
| Yes (Female) | 0 | | 0 | | 1 | (2.3%) | 4 | (8.5%) |
| No (Female) | 14 | (100%) | 11 | (100%) | 42 | (97.7%) | 43 | (91.5%) |
| Diabetic retinopathy | | | | | | | | |
| [n (%)] | | | | | | | | |
| Number | 34 | | 30 | | 127 | | 130 | |
| Yes | 5 | (14.7%) | 4 | (13.3%) | 23 | (18.1%) | 28 | (21.5%) |
| Photocoagulation performed: | 1 | (2.9%) | 2 | (6.7%) | 6 | (4.7%) | 5 | (3.8%) |
| Yes | | | | | | | | |
| Photocoagulation performed: | 4 | (11.8%) | 2 | (6.7%) | 17 | (13.4%) | 23 | (17.7%) |
| No | | | | | | | | |
| Vitrectomy performed because | 1 | (2.9%) | 1 | (3.3%) | 2 | (1.6%) | 1 | (0.8%) |
| of diabetic retinopathy: Yes | | | | | | | | |
| Vitrectomy performed because | 4 | (11.8%) | 3 | (10.0%) | 21 | (16.5%) | 27 | (20.8%) |
| of diabetic retinopathy: No | | | | | | | | |
| No | 29 | (85.3%) | 26 | (86.7%) | 102 | (80.3%) | 100 | (76.9%) |
| Unknown | 0 | | 0 | | 2 | (1.6%) | 2 | (1.5%) |
| Diabetic sensory or motor neuropathy | | | | | | | | |
| [n (%)] | | | | | | | | |
| Number | 34 | | 30 | | 127 | | 130 | |
| Yes | 5 | (14.7%) | 4 | (13.3%) | 16 | (12.6%) | 17 | (13.1%) |
| No | 25 | (73.5%) | 25 | (83.3%) | 106 | (83.5%) | 109 | (83.8%) |
| Unknown | 4 | (11.8%) | 1 | (3.3%) | 5 | (3.9%) | 4 | (3.1%) |
| Diabetic autonomic neuropathy | | | | | | | | |
| [n (%)] | | | | | | | | |
| Number | 34 | | 30 | | 127 | | 130 | |
| Yes | 1 | (2.9%) | 1 | (3.3%) | 3 | (2.4%) | 2 | (1.5%) |
| No | 29 | (85.3%) | 28 | (93.3%) | 117 | (92.1%) | 123 | (94.6%) |
| Unknown | 4 | (11.8%) | 1 | (3.3%) | 7 | (5.5%) | 5 | (3.8%) |
| Diabetic nephropathy | | | | | | | | |
| [n (%)] | | | | | | | | |
| Number | 34 | | 30 | | 127 | | 130 | |
| Yes | 10 | (29.4%) | 6 | (20.0%) | 34 | (26.8%) | 36 | (27.7%) |
| Impaired renal function | 0 | | 0 | | 3 | (2.4%) | 2 | (1.5%) |
| (estimated GFR by MDRD | | | | | | | | |
| below 60 ml/min) | | | | | | | | |
| Microalbuminuria (30 to 299 | 7 | (20.6%) | 6 | (20.0%) | 27 | (21.3%) | 30 | (23.1%) |
| mcg per mg creatinine) | | | | | | | | |
| Overt proteinuria (equal to or | 3 | (8.8%) | 0 | | 4 | (3.1%) | 4 | (3.1%) |
| above 300 mcg per mg | | | | | | | | |
| creatinine) | | | | | | | | |
| No | 24 | (70.6%) | 24 | (80.0%) | 92 | (72.4%) | 92 | (70.8%) |
| Unknown | 0 | | 0 | | 1 | (0.8%) | 2 | (1.5%) |
| eGFR at screening | | | | | | | | |
| (ml/min/1.73 m²) | | | | | | | | |
| Number | 34 | | 30 | | 127 | | 130 | |
| Mean (SD) | 115.35 | (27.23) | 128.84 | (28.36) | 106.86 | (26.98) | 108.92 | (30.24) |
| Median | 112.20 | | 125.75 | | 104.60 | | 107.25 | |
| Min:Max | 64.3:210.7 | | 77.0:184.8 | | 57.9:180.2 | | 47.0:229.6 | |
| eGFR categories at screening | | | | | | | | |
| (ml/min/1.73 m²) | | | | | | | | |
| [n (%)] | | | | | | | | |
| Number | 34 | | 30 | | 127 | | 130 | |
| <15 (end stage renal disease) | 0 | | 0 | | 0 | | 0 | |
| ≥15 to <30 (severe decrease in | 0 | | 0 | | 0 | | 0 | |
| GFR) | | | | | | | | |
| ≥30 to <60 (moderate decrease in | 0 | | 0 | | 1 | (0.8%) | 4 | (3.1%) |
| GFR) | | | | | | | | |
| ≥60 to <90 (mild decrease in GFR) | 4 | (11.8%) | 4 | (13.3%) | 33 | (26.0%) | 30 | (23.1%) |
| ≥90 (normal) | 30 | (88.2%) | 26 | (86.7%) | 93 | (73.2%) | 96 | (73.8%) |

GLP-1 = Glucagon like peptide-1,
GFR = Glomerular filtration rate,
eGFR = Estimated glomerular filtration rate,
SGLT2 = Sodium glucose co-transporter 2.
eGFR value is derived using the equation of IDMS-MDRD formula.

TABLE 3
Mean change in HbA1c (%) from baseline to Week 26 during the 26-week on-treatment period by
SGLT2 inhibitor use at screening-mITT population (EFC14112)

| | | Fixed Ratio Combination | | | | |
|---|---|---|---|---|---|---|
| | | (N = 161) | | | | |
| | | | Compared to Lixisenatide | | Lixisenatide | |
| | | | Difference in | | (N = 160) | |
| | N | LSMean (SE)a | LSMean (SE)a | 95% CIa | N | LSMean (SE)a |
| SGLT2 inhibitor use | | | | | | |
| at screening | | | | | | |
| Yes | 34 | −1.39 (0.148) | −1.00 (0.217) | (−1.429 to −0.573) | 30 | −0.39 (0.161) |
| No | 127 | −1.66 (0.077) | −1.11 (0.110) | (−1.325 to −0.890) | 127 | −0.55 (0.081) |

SGLT2 = Sodium glucose co-transporter 2.
aMixed-effect model with repeated measures (MMRM) with treatment groups (fixed ratio combination, lixisenatide), randomization strata of HbA1c (<8.0%, ≥8.0%) at Visit 1 (Week-2), randomization strata of DPP-4 inhibitor use at screening (Yes, No), visit (Week 4, 8, 12, 16, 20, and 26), subgroup factor, treatment-by-vist, treatment-by-subgroup factor, visit-by-subgroup factor, treatment-by-visit-by-subgroup factor as fixed effects, and baseline HbA1c value-by-visit interaction as a covariate.
The analysis included measurements obtained before the introduction of rescue medication and up to 14 days after the last injection of open-label investigational medicinal product on or before Visit 16 (Week 26), or Day 183 if Visit 16 (Week 26) is not available.
Included are patients who have measurements at baseline and post-baseline.
LS Mean and difference in LS Mean were provided for categories with ≥5 patients in each treatment group.

TABLE 4
Mean change in fasting plasma glucose (mmol/L) from baseline to Week 26 during the 26-week
on-treatment period by SGLT2 inhibitor use at screening-mITT population (EFC14112)

| | Fixed Ratio Combination | | | | Lixisenatide | |
|---|---|---|---|---|---|---|
| | (N = 161) | | | | (N = 160) | |
| | Compared to Lixisenatide | | | | | |
| | | | Difference in | | | |
| | N | LSMean (SE)a | LSMean (SE)a | 95% CIa | N | LSMean (SE)a |
| SGLT2 inhibitor use | | | | | | |
| at screening | | | | | | |
| Yes | 34 | −1.75 (0.287) | −2.16 (0.415) | (−2.977 to −1.343) | 30 | 0.41 (0.318) |
| No | 127 | −2.48 (0.150) | −2.23 (0.212) | (−2.647 to −1.813) | 125 | −0.25 (0.160) |

SGLT2 = Sodium glucose co-transporter 2.
aMixed-effect model with repeated measures (MMRM) with treatment groups (fixed ratio combination, lixisenatide), randomization strata of HbA1c (<8.0%, ≥8.0%) at Visit 1 (Week-2), randomization strata of DPP-4 inhibitor use at screening (Yes, No), visit (Week 4, 8, 12, 16, 20, and 26), subgroup factor, treatment-by-vist, treatment-by-subgroup factor, visit-by-subgroup factor, treatment-by-visit-by-subgroup factor as fixed effects, and baseline fasting plasma glucose value-by-visit interaction as a covariate.
The analysis included measurements obtained before the introduction of rescue medication and up to 1 day after the last injection of open-label investigational medicinal product on or before Visit 16 (Week 26), or Day 183 if Visit 16 (Week 26) is not available.
Included are patients who have measurements at baseline and post-baseline.
LS Mean and difference in LS Mean were provided for categories with ≥5 patients in each treatment group.

TABLE 5
Overview of adverse event profile: treatment emergent adverse events by SGLT-2 inhibitor
use at screening during the 26-week on-treatment period-Safety population (EFC14112)

| | SGLT2 inhibitor use at screening | | SGLT2 inhibitor use at screening | |
|---|---|---|---|---|
| | Yes | | No | |
| | (N = 64) | | (N = 257) | |
| | Fixed Ratio | | Fixed Ratio | |
| | Combination | Lixisenatide | Combination | Lixisenatide |
| n (%) | (N = 34) | (N = 30) | (N = 127) | (N = 130) |
| Patients with any TEAE | 24 (70.6%) | 24 (80.0%) | 85 (66.9%) | 98 (75.4%) |
| Patients with any serious TEAE | 1 (2.9%) | 0 | 4 (3.1%) | 4 (3.1%) |
| Patients with any TEAE leading to | 0 | 0 | 0 | 1 (0.8%) |
| death | | | | |
| Patients with any TEAE leading to | 0 | 1 (3.3%) | 4 (3.1%) | 16 (12.3%) |
| permanent treatment | | | | |
| discontinuation | | | | |

SGLT2 = Sodium glucose co-transporter 2,
TEAE: Treatment emergent adverse event.
n (%) = number and percentage of patients with at least one TEAE.
The 26-week on-treatment period is defined as the time from the first injection of open-label investigational medicinal product (IMP) up to 3 days after the last injection of open-label IMP on or before Week 26 visit (or Day 183 if Week 26 visit is missing), regardless of the introduction of rescue therapy.

TABLE 6
Number (%) of patients experiencing common TEAE(s) (PT >=2% in any treatment
group) presented by primary SOC and PT and by SGLT-2 inibitor use at screening
during the 26-week on-treatment period-Safety population (EFC14112)

| | SGLT2 inhibitor use at screening | | | | SGLT2 inhibitor use at screening | | | |
|---|---|---|---|---|---|---|---|---|
| | Yes | | | | No | | | |
| Primary System | (N = 64) | | | | (N = 257) | | | |
| Organ Class | Fixed Ratio | | | | Fixed Ratio | | | |
| Preferred Term | Combination | | Lixisenatide | | Combination | | Lixisenatide | |
| n (%) | (N = 34) | | (N = 30) | | (N = 127) | | (N = 130) | |
| Any class | 24 | (70.6%) | 24 | (80.0%) | 85 | (66.9%) | 98 | (75.4%) |
| Infections and infestations | 14 | (41.2%) | 15 | (50.0%) | 48 | (37.8%) | 48 | (36.9%) |
| Nasopharyngitis | 6 | (17.6%) | 6 | (20.0%) | 32 | (25.2%) | 29 | (22.3%) |
| Conjunctivitis | 1 | (2.9%) | 0 | | 4 | (3.1%) | 0 | |
| Gastroenteritis | 2 | (5.9%) | 1 | (3.3%) | 3 | (2.4%) | 5 | (3.8%) |
| Bronchitis | 0 | | 3 | (10.0%) | 4 | (3.1%) | 3 | (2.3%) |
| Cystitis | 1 | (2.9%) | 2 | (6.7%) | 1 | (0.8%) | 2 | (1.5%) |
| Influenza | 0 | | 1 | (3.3%) | 0 | | 6 | (4.6%) |
| Metabolism and nutrition disorders | 0 | | 1 | (3.3%) | 8 | (6.3%) | 6 | (4.6%) |
| Decreased appetite | 0 | | 1 | (3.3%) | 5 | (3.9%) | 4 | (3.1%) |
| Nervous system disorders | 2 | (5.9%) | 3 | (10.0%) | 17 | (13.4%) | 8 | (6.2%) |
| Somnolence | 1 | (2.9%) | 1 | (3.3%) | 5 | (3.9%) | 0 | |
| Dizziness | 0 | | 0 | | 4 | (3.1%) | 2 | (1.5%) |
| Gastrointestinal disorders | 6 | (17.6%) | 11 | (36.7%) | 41 | (32.3%) | 57 | (43.8%) |
| Nausea | 0 | | 5 | (16.7%) | 23 | (18.1%) | 38 | (29.2%) |
| Vomiting | 1 | (2.9%) | 1 | (3.3%) | 8 | (6.3%) | 7 | (5.4%) |
| Diarrhoea | 0 | | 2 | (6.7%) | 8 | (6.3%) | 8 | (6.2%) |
| Abdominal discomfort | 1 | (2.9%) | 0 | | 6 | (4.7%) | 2 | (1.5%) |
| Dyspepsia | 2 | (5.9%) | 5 | (16.7%) | 2 | (1.6%) | 4 | (3.1%) |
| Constipation | 1 | (2.9%) | 2 | (6.7%) | 1 | (0.8%) | 7 | (5.4%) |
| Skin and subcutaneous tissue | 4 | (11.8%) | 3 | (10.0%) | 12 | (9.4%) | 8 | (6.2%) |
| disorders | | | | | | | | |
| Eczema | 1 | (2.9%) | 2 | (6.7%) | 3 | (2.4%) | 1 | (0.8%) |
| Musculoskeletal and connective | 5 | (14.7%) | 5 | (16.7%) | 11 | (8.7%) | 10 | (7.7%) |
| tissue disorders | | | | | | | | |
| Back pain | 2 | (5.9%) | 0 | | 3 | (2.4%) | 5 | (3.8%) |
| General disorders and administration | 1 | (2.9%) | 2 | (6.7%) | 12 | (9.4%) | 17 | (13.1%) |
| site conditions | | | | | | | | |
| Injection site reaction | 1 | (2.9%) | 1 | (3.3%) | 0 | | 3 | (2.3%) |
| Injury, poisoning and procedural | 4 | (11.8%) | 1 | (3.3%) | 10 | (7.9%) | 10 | (7.7%) |
| complications | | | | | | | | |

| | | | | | | | |
|---|---|---|---|---|---|---|---|
| Ligament sprain | 2 | (5.9%) | 0 | 5 | (3.9%) | 3 | (2.3%) |

| | | | | | | | |
|---|---|---|---|---|---|---|---|
| Contusion | 0 | 1 | (3.3%) | 2 | (1.6%) | 4 | (3.1%) |

SGLT2 = Sodium glucose co-transporter 2,
TEAE: Treatment emergent adverse event,
SOC: System organ class,
PT: Preferred term.
MedDRA 20.1.
n (%) = number and percentage of patients with at least one TEAE.
Note:
Table sorted by SOC internationally agreed order and decreasing frequency of PT sorted by decreasing frequency within a SOC according to all TEAE summary.
Only SOC with at least one PT ≥2% in at least one treatment group are presented.
The 26-week on-treatment period is defined as the time from the first injection of open-label investigational medicinal product (IMP) up to 3 days after the last injection of open-label IMP on or before Week 26 visit (or Day 183 if Week 26 visit is missing), regardless of the introduction of rescue therapy.

TABLE 7
Summary of documented symptomatic hypoglycemia recorded on the dedicated eCRF
page and meeting protocol definition by SGLT2 inhibitor use at screening
during the 26-week on- treatment period-Safety population (EFC14112)

| | SGLT2 inhibitor use at screening | | SGLT2 inhibitor use at screening | |
|---|---|---|---|---|
| | Yes | | No | |
| | (N = 64) | | (N = 257) | |
| | Fixed Ratio | | Fixed Ratio | |
| | Combination | Lixisenatide | Combination | Lixisenatide |
| | (N = 34) | (N = 30) | (N = 127) | (N = 130) |
| Total patient years of exposure | 17.1 | 14.9 | 63.1 | 59.5 |
| Documented symptomatic | | | | |
| hypoglycemia (plasma glucose ≤70 | | | | |
| mg/dL [3.9 mmol/L]) | | | | |
| Number of patients with events, n | 2 (5.9%) | 1 (3.3%) | 19 (15.0%) | 3 (2.3%) |
| (%) | | | | |
| Number of patients with events per | 0.12 | 0.07 | 0.30 | 0.05 |
| patient yeara | | | | |
| Number of events | 3 | 1 | 72 | 5 |
| Number of events per patient yearb | 0.18 | 0.07 | 1.14 | 0.08 |

SGLT2 = Sodium glucose co-transporter 2,
IMP = Investigational medicinal product,
eCRF = Electronic case report form.
Total patient years of exposure: calculated as the time from the first injection of open-label IMP up to 1 day after the last injection of open-label IMP during the 26-week treatment period.
aCalculated as number of patients with events divided by total patient years of exposure.
bCalculated as number of events divided by total patient years of exposure.
The 26-week on-treatment period is defined as the time from the first injection of open-label investigational medicinal product (IMP) up to 1 day after the last injection of open-label IMP on or before Week 26 visit (or Day 183 if Week 26 visit is missing), regardless of the introduction of rescue therapy.

### Example 6

Concomitant Use of a Fixed-Dose Ratio Formulation of Insulin Glargine and Lixisenatide with SGLT2i—Subgroup Analysis of Example 3

The concomitant use of a fixed-dose ratio combination (FRC) of insulin glargine and lixisenatide with an SGLT2 inhibitor (SGLT2i) is supported by subgroup analyses of the patient group being subject of Example 3 (study EFC14114). In this study, 59 patients (22.7%) received concomitantly the FRC and a SGLT2i.

The FRC was provided in a single formulation, as described in Example 3.

Efficacy results (change from baseline to Week 26 in HbA1c, FPG and 2-hour PPG) in both treatment groups were generally similar in SGLT2i users and non-users (Table 3, Table 4, Table 5). In particular, there was no indication of a decreased efficacy of the FRC in the SGLT2i user subgroup.

Overview of safety did not reveal differences between SGLT2i users and non-users (Table 6). With regard to common TEAEs (Table 7), TEAEs in the gastrointestinal SOC were also reported numerically less frequently in the FRC group in SGLT2i users when compared to SGLT2i non-users (22.0% versus 27.4%, respectively). No new safety signal was identified when patients in the FRC treatment group were using SGLT2i as background therapy compared to SGLT2i non-users.

TABLE 1
Demographics and patient characteristics at screening or baseline by
SGLT2 inhibitor use at screening-Randomized population (EFC14114)

| | SGLT2 inhibitor use at screening | | | | SGLT2 inhibitor use at screening | | | |
|---|---|---|---|---|---|---|---|---|
| | Yes | | | | No | | | |
| | (N = 115) | | | | (N = 406) | | | |
| | Fixed Ratio | | Insulin | | Fixed Ratio | | Insulin | |
| | Combination | | glargine | | Combination | | glargine | |
| | (N = 59) | | (N = 56) | | (N = 201) | | (N = 205) | |
| Age (years) | | | | | | | | |
| Number | 59 | | 56 | | 201 | | 205 | |
| Mean (SD) | 53.8 | (10.8) | 56.4 | (11.2) | 60.8 | (10.6) | 61.2 | (9.8) |
| Median | 53.0 | | 57.0 | | 62.0 | | 62.0 | |
| Min:Max | 27:79 | | 34:80 | | 30:81 | | 40:82 | |
| Age group (years) | | | | | | | | |
| [n (%)] | | | | | | | | |
| Number | 59 | | 56 | | 201 | | 205 | |
| <65 | 48 | (81.4%) | 40 | (71.4%) | 120 | (59.7%) | 121 | (59.0%) |
| ≥65 to <75 | 9 | (15.3%) | 14 | (25.0%) | 66 | (32.8%) | 68 | (33.2%) |
| ≥75 | 2 | (3.4%) | 2 | (3.6%) | 15 | (7.5%) | 16 | (7.8%) |
| Gender | | | | | | | | |
| [n (%)] | | | | | | | | |
| Number | 59 | | 56 | | 201 | | 205 | |
| Male | 40 | (67.8%) | 34 | (60.7%) | 134 | (66.7%) | 133 | (64.9%) |
| Female | 19 | (32.2%) | 22 | (39.3%) | 67 | (33.3%) | 72 | (35.1%) |
| Race | | | | | | | | |
| [n (%)] | | | | | | | | |
| Number | 59 | | 56 | | 201 | | 205 | |
| Asian | 59 | (100%) | 56 | (100%) | 201 | (100%) | 205 | (100%) |
| HbA1c (%) at screening | | | | | | | | |
| Number | 59 | | 56 | | 201 | | 205 | |
| Mean (SD) | 8.25 | (0.55) | 8.18 | (0.47) | 8.15 | (0.51) | 8.10 | (0.46) |
| Median | 8.20 | | 8.10 | | 8.10 | | 8.00 | |
| Min:Max | 7.5:9.4 | | 7.5:9.4 | | 7.5:9.5 | | 7.5:9.5 | |
| HbA1c (%) categories at screening | | | | | | | | |
| [n (%)] | | | | | | | | |
| Number | 59 | | 56 | | 201 | | 205 | |
| <8 | 21 | (35.6%) | 19 | (33.9%) | 89 | (44.3%) | 90 | (43.9%) |
| ≥8 | 38 | (64.4%) | 37 | (66.1%) | 112 | (55.7%) | 115 | (56.1%) |
| Randomization strata of HbA1c | | | | | | | | |
| (%) from IVRS/IWRS | | | | | | | | |
| Number | 59 | | 56 | | 201 | | 205 | |
| <8 | 21 | (35.6%) | 20 | (35.7%) | 89 | (44.3%) | 90 | (43.9%) |
| ≥8 | 38 | (64.4%) | 36 | (64.3%) | 112 | (55.7%) | 115 | (56.1%) |
| Randomization strata of DPP-4 | | | | | | | | |
| inhibitor use at screening | | | | | | | | |
| from IVRS/IWRS | | | | | | | | |
| Number | 59 | | 56 | | 201 | | 205 | |
| Yes | 16 | (27.1%) | 18 | (32.1%) | 114 | (56.7%) | 115 | (56.1%) |
| No | 43 | (72.9%) | 38 | (67.9%) | 87 | (43.3%) | 90 | (43.9%) |
| Baseline BMI | | | | | | | | |
| (kg/m²) | | | | | | | | |
| Number | 59 | | 56 | | 201 | | 205 | |
| Mean (SD) | 27.68 | (4.18) | 26.84 | (4.36) | 25.76 | (4.25) | 25.61 | (4.29) |
| Median | 27.04 | | 26.00 | | 25.38 | | 25.39 | |
| Min:Max | 20.2:37.8 | | 20.1:38.6 | | 17.6:39.7 | | 17.0:39.3 | |
| Baseline BMI categories | | | | | | | | |
| (kg/m²) | | | | | | | | |
| [n (%)] | | | | | | | | |
| Number | 59 | | 56 | | 201 | | 205 | |
| <25 | 17 | (28.8%) | 21 | (37.5%) | 94 | (46.8%) | 96 | (46.8%) |
| ≥25 to <30 | 26 | (44.1%) | 23 | (41.1%) | 73 | (36.3%) | 80 | (39.0%) |
| ≥30 | 16 | (27.1%) | 12 | (21.4%) | 34 | (16.9%) | 29 | (14.1%) |
| FPG(mmol/L) at screening | | | | | | | | |
| Number | 59 | | 56 | | 201 | | 205 | |
| Mean (SD) | 8.25 | (0.89) | 8.60 | (0.99) | 8.65 | (0.87) | 8.74 | (0.93) |
| Median | 8.22 | | 8.85 | | 8.77 | | 8.83 | |
| Min:Max | 5.8:10.0 | | 6.3:10.0 | | 5.4:10.0 | | 4.7:10.5 | |
| FPG(mg/dL) at screening | | | | | | | | |
| Number | 59 | | 56 | | 201 | | 205 | |
| Mean (SD) | 148.7 | (16.1) | 155.0 | (17.9) | 155.9 | (15.6) | 157.5 | (16.7) |

| | | | | |
|---|---|---|---|---|
| Median | 148.0 | 159.5 | 158.0 | 159.0 |
| Min:Max | 105:180 | 114:180 | 97:180 | 85:189 |

SGLT2 = Sodium glucose co-transporter 2,
BMI = Body Mass Index,
FPG = Fasting plasma glucose,
IVRS/IWRS = Interactive Voice/Web Response System.

TABLE 2
Disease characteristics at screening or baseline by SGLT2 inhibitor
use at screening-Randomized population (EFC14114)

| | SGLT2 inhibitor use at screening | | | | SGLT2 inhibitor use at screening | | | |
|---|---|---|---|---|---|---|---|---|
| | Yes | | | | No | | | |
| | (N = 115) | | | | (N = 406) | | | |
| | Fixed Ratio | | Insulin | | Fixed Ratio | | Insulin | |
| | Combination | | glargine | | Combination | | glargine | |
| | (N = 59) | | (N = 56) | | (N = 201) | | (N = 205) | |
| Duration of diabetes | | | | | | | | |
| (years) | | | | | | | | |
| Number | 59 | | 56 | | 201 | | 205 | |
| Mean (SD) | 6.46 | (4.99) | 7.76 | (5.45) | 9.59 | (6.72) | 10.12 | (6.98) |
| Median | 5.25 | | 6.60 | | 8.01 | | 8.45 | |
| Min:Max | 1.0:25.2 | | 1.1:26.4 | | 1.0:32.5 | | 1.0:41.4 | |
| Age at onset of Type 2 diabetes | | | | | | | | |
| (years) | | | | | | | | |
| Number | 59 | | 56 | | 201 | | 205 | |
| Mean (SD) | 47.3 | (10.5) | 48.6 | (11.4) | 51.2 | (10.1) | 51.1 | (10.3) |
| Median | 45.0 | | 48.5 | | 52.0 | | 52.0 | |
| Min:Max | 26:78 | | 20:69 | | 14:74 | | 25:76 | |
| Prior use of insulin | | | | | | | | |
| [n (%)] | | | | | | | | |
| Number | 59 | | 56 | | 201 | | 205 | |
| Yes | 2 | (3.4%) | 3 | (5.4%) | 17 | (8.5%) | 15 | (7.3%) |
| No | 57 | (96.6%) | 53 | (94.6%) | 184 | (91.5%) | 190 | (92.7%) |
| Prior use of GLP-1 receptor agonist | | | | | | | | |
| [n (%)] | | | | | | | | |
| Number | 59 | | 56 | | 201 | | 205 | |
| Yes | 10 | (16.9%) | 12 | (21.4%) | 35 | (17.4%) | 36 | (17.6%) |
| No | 49 | (83.1%) | 44 | (78.6%) | 166 | (82.6%) | 169 | (82.4%) |
| History of gestational diabetes | | | | | | | | |
| [n (%)] | | | | | | | | |
| Number (Female) | 19 | | 22 | | 67 | | 72 | |
| Yes (Female) | 1 | (5.3%) | 0 | | 3 | (4.5%) | 1 | (1.4%) |
| No (Female) | 18 | (94.7%) | 22 | (100%) | 64 | (95.5%) | 71 | (98.6%) |
| Diabetic retinopathy | | | | | | | | |
| [n (%)] | | | | | | | | |
| Number | 59 | | 56 | | 201 | | 205 | |
| Yes | 6 | (10.2%) | 11 | (19.6%) | 35 | (17.4%) | 46 | (22.4%) |
| Photocoagulation performed: Yes | 1 | (1.7%) | 3 | (5.4%) | 5 | (2.5%) | 7 | (3.4%) |
| Photocoagulation performed: No | 5 | (8.5%) | 8 | (14.3%) | 29 | (14.4%) | 39 | (19.0%) |
| Vitrectomy performed because of | 0 | | 0 | | 1 | (0.5%) | 3 | (1.5%) |
| diabetic retinopathy: Yes | | | | | | | | |
| Vitrectomy performed because of | 6 | (10.2%) | 11 | (19.6%) | 34 | (16.9%) | 43 | (21.0%) |
| diabetic retinopathy: No | | | | | | | | |
| No | 50 | (84.7%) | 43 | (76.8%) | 160 | (79.6%) | 154 | (75.1%) |
| Unknown | 3 | (5.1%) | 2 | (3.6%) | 6 | (3.0%) | 5 | (2.4%) |
| Diabetic sensory or motor neuropathy | | | | | | | | |
| [n (%)] | | | | | | | | |
| Number | 59 | | 56 | | 201 | | 205 | |
| Yes | 7 | (11.9%) | 6 | (10.7%) | 35 | (17.4%) | 31 | (15.1%) |
| No | 49 | (83.1%) | 49 | (87.5%) | 159 | (79.1%) | 168 | (82.0%) |
| Unknown | 3 | (5.1%) | 1 | (1.8%) | 7 | (3.5%) | 6 | (2.9%) |
| Diabetic autonomic neuropathy | | | | | | | | |
| [n (%)] | | | | | | | | |
| Number | 59 | | 56 | | 201 | | 205 | |
| Yes | 3 | (5.1%) | 1 | (1.8%) | 7 | (3.5%) | 12 | (5.9%) |
| No | 52 | (88.1%) | 54 | (96.4%) | 184 | (91.5%) | 186 | (90.7%) |
| Unknown | 4 | (6.8%) | 1 | (1.8%) | 10 | (5.0%) | 7 | (3.4%) |
| Diabetic nephropathy | | | | | | | | |
| [n (%)] | | | | | | | | |
| Number | 59 | | 56 | | 201 | | 205 | |
| Yes | 7 | (11.9%) | 6 | (10.7%) | 52 | (25.9%) | 43 | (21.0%) |
| Impaired renal function (estimated | 1 | (1.7%) | 1 | (1.8%) | 6 | (3.0%) | 4 | (2.0%) |
| GFR by MDRD below 60 ml/min) | | | | | | | | |
| Microalbuminuria (30 to 299 mcg per | 5 | (8.5%) | 4 | (7.1%) | 43 | (21.4%) | 30 | (14.6%) |
| mg creatinine) | | | | | | | | |
| Overt proteinuria (equal to or above | 1 | (1.7%) | 1 | (1.8%) | 4 | (2.0%) | 9 | (4.4%) |
| 300 mcg per mg creatinine) | | | | | | | | |
| No | 49 | (83.1%) | 48 | (85.7%) | 144 | (71.6%) | 160 | (78.0%) |
| Unknown | 3 | (5.1%) | 2 | (3.6%) | 5 | (2.5%) | 2 | (1.0%) |
| eGFR at screening | | | | | | | | |
| (ml/min/1.73 m²) | | | | | | | | |
| Number | 59 | | 56 | | 201 | | 205 | |
| Mean (SD) | 116.76 | (30.95) | 113.19 | (32.47) | 104.58 | (26.50) | 106.93 | (29.36) |
| Median | 112.30 | | 107.65 | | 103.90 | | 104.90 | |
| Min:Max | 46.3:196.7 | | 53.9:209.3 | | 55.9:229.5 | | 50.3:335.7 | |
| eGFR categories at screening | | | | | | | | |
| (ml/min/1.73 m²) | | | | | | | | |
| [n (%)] | | | | | | | | |
| Number | 59 | | 56 | | 201 | | 205 | |
| <15 (end stage renal disease) | 0 | | 0 | | 0 | | 0 | |
| ≥15 to <30 (severe decrease in GFR) | 0 | | 0 | | 0 | | 0 | |
| ≥30 to <60 (moderate decrease in GFR) | 1 | (1.7%) | 2 | (3.6%) | 3 | (1.5%) | 3 | (1.5%) |
| ≥60 to <90 (mild decrease in GFR) | 7 | (11.9%) | 11 | (19.6%) | 59 | (29.4%) | 51 | (24.9%) |
| ≥90 (normal) | 51 | (86.4%) | 43 | (76.8%) | 139 | (69.2%) | 151 | (73.7%) |

SGLT2 = Sodium glucose co-transporter 2,
GLP-1 = Glucagon like peptide-1.
GFR = Glomerular filtration rate,
eGFR = Estimated glomerular filtration rate.
eGFR value is derived using the equation of IDMS-MDRD formula.

TABLE 3
Mean change in HbA1c (%) from baseline to Week 26 during the on-treament period by
SGLT2 inhibitor use at screening-mITT population (EFC14114)

| | | Fixed Ratio Combination | | | | |
|---|---|---|---|---|---|---|
| | | (N = 260) | | | | |
| | | | Compared to Insulin Glargine | | Insulin Glargine | |
| | | | Difference in | | (N = 260) | |
| | N | LSMean (SE)a | LSMean (SE)a | 95% CIa | N | LSMean (SE)a |
| SGLT2 inhibitor use | | | | | | |
| at screening | | | | | | |
| Yes | 58 | −1.19 (0.088) | −0.70 (0.126) | (−0.952 to −0.455) | 55 | −0.49 (0.091) |
| No | 199 | −1.48 (0.047) | −0.65 (0.067) | (−0.780 to −0.518) | 205 | −0.83 (0.047) |

SGLT2 = Sodium glucose co-transporter 2.
aMixed-effect model with repeated measures (MMRM) with treatment groups (fixed ratio combination, insulin glargine), randomization strata of HbA1c (<8.0%, ≥8.0%) at Visit 1 (Week-2), randomization strata of DPP-4 inhibitor use at screening (Yes, No), visit (Week 4, 8, 12, 16, 20, and 26), subgroup factor, treatment-by-vist, treatment-by-subgroup factor, visit-by-subgroup factor, treatment-by-visit-by-subgroup factor as fixed effects, and baseline HbA1c value-by-visit interaction as a covariate.
The analysis excluded measurements obtained after the introduction of rescue medication and/or after the treatment cessation plus 14 days.
Included are patients who have measurements at baseline and post-baseline.
LS Mean and difference in LS Mean were provided for categories with ≥5 patients in each treatment group.

TABLE 4
Mean change in fasting plasma glucose (mmol/L) from baseline to Week 26 during
the on-treatment period by SGLT2 inhibitor use at screening-mITT population (EFC14114)

| | | Fixed Ratio Combination | | | | |
|---|---|---|---|---|---|---|
| | | (N = 260) | | | | |
| | | | Compared to Insulin Glargine | | Insulin Glargine | |
| | | | Difference in | | (N = 260) | |
| | N | LSMean (SE)a | LSMean (SE)a | 95% CIa | N | LSMean (SE)a |
| SGLT2 inhibitor use | | | | | | |
| at screening | | | | | | |
| Yes | 58 | −1.24 (0.198) | −0.23 (0.283) | (−0.785 to 0.327) | 55 | −1.01 (0.205) |
| No | 198 | −1.93 (0.106) | −0.47 (0.149) | (−0.759 to −0.175) | 205 | −1.46 (0.104) |

SGLT2 = Sodium glucose co-transporter 2.
aMixed-effect model with repeated measures (MMRM) with treatment groups (fixed ratio combination, insulin glargine), randomization strata of HbA1c (<8.0%, ≥8.0%) at Visit 1 (Week-2), randomization strata of DPP-4 inhibitor use at screening (Yes, No), visit (Week 4, 8, 12, 16, 20, and 26), subgroup factor, treatment-by-vist, treatment-by-subgroup factor, visit-by-subgroup factor, treatment-by-visit-by-subgroup factor as fixed effects, and baseline fasting plasma glucose value-by-visit interaction as a covariate.
The analysis excluded measurements obtained after the introduction of rescue medication and/or after the treatment cessation plus 1 day.
Included are patients who have measurements at baseline and post-baseline.
LS Mean and difference in LS Mean were provided for categories with ≥5 patients in each treatment group.

TABLE 5
Mean change in 2-hour postprandial plasma glucose (mmol/L) during a standardized meal
test from baseline to Week 26 during the on-treatment period by
SGLT2 inhibitor use at screening-mITT population (EFC14114)

| | | Fixed Ratio Combination | | | | |
|---|---|---|---|---|---|---|
| | | (N = 260) | | | | |
| | | | Compared to Insulin Glargine | | Insulin Glargine | |
| | | | Difference in | | (N = 260) | |
| | N | LSMean (SE)a | LSMean (SE)a | 95% CIa | N | LSMean (SE)a |
| SGLT2 inhibitor use | | | | | | |
| at screening | | | | | | |
| Yes | 57 | −5.56 (0.350) | −4.18 (0.495) | (−5.149 to −3.202) | 53 | −1.38 (0.363) |
| No | 191 | −6.24 (0.189) | −4.95 (0.263) | (−5.470 to −4.439) | 199 | −1.29 (0.185) |

SGLT2 = Sodium glucose co-transporter 2.
aAnalysis of covariance (ANCOVA) model with treatment groups (fixed ratio combination, insulin glargine), randomization strata of HbA1c (<8.0%, ≥8.0%) at Visit 1 (Week-2), randomization strata of DPP-4 inhibitor use at screening (Yes, No), subgroup factor and treatment-by-subgroup factor as fixed effects, and baseline 2-hour postprandial plasma glucose value-by-visit interaction as a covariate.
For fixied ratio combination group, the analysis excluded measurements obtained after the introduction of rescue medication and/or after the treatment cessation.
For insulin glargine group, the analysis excluded measurements obtained after the introduction of rescue medication and/or after the treatment cessation plus 1 day.
Patients with both baseline and Week 26 (LOCF) measurements were included.
LS Mean and difference in LS Mean were provided for categories with ≥5 patients in each treatment group.

TABLE 6
Overview of adverse event profile: treatment emergent adverse events
by SGLT-2 inhibitor use at screening-Safety population (EFC14114)

| | SGLT2 inhibitor use at screening | | SGLT2 inhibitor use at screening | |
|---|---|---|---|---|
| | Yes | | No | |
| | (N = 115) | | (N = 406) | |
| | Fixed Ratio | Insulin | Fixed Ratio | Insulin |
| | Combination | glargine | Combination | glargine |
| n (%) | (N = 59) | (N = 56) | (N = 201) | (N = 205) |
| Patients with any TEAE | 41 (69.5%) | 36 (64.3%) | 122 (60.7%) | 130 (63.4%) |
| Patients with any serious TEAE | 1 (1.7%) | 1 (1.8%) | 5 (2.5%) | 7 (3.4%) |
| Patients with any TEAE leading | 0 | 0 | 1 (0.5%) | 0 |
| to death | | | | |
| Patients with any TEAE leading | 1 (1.7%) | 1 (1.8%) | 3 (1.5%) | 1 (0.5%) |
| to permanent treatment discontinuation | | | | |

SGLT2 = Sodium glucose co-transporter 2,
TEAE: Treatment emergent adverse event,
n (%) = number and percentage of patients with at least one TEAE.

TABLE 7
Number (%) of patients experiencing common TEAE(s) (PT >=2% in any treatment group)
by primary SOC and PT and by SGLT-2 inibitor use at screening-Safety population (EFC14114)

| | SGLT2 inhibitor use at screening | | | | SGLT2 inhibitor use at screening | | | |
|---|---|---|---|---|---|---|---|---|
| | Yes | | | | No | | | |
| Primary System | (N = 115) | | | | (N = 406) | | | |
| Organ Class | Fixed Ratio | | Insulin | | Fixed Ratio | | Insulin | |
| Preferred Term | Combination | | glargine | | Combination | | glargine | |
| n (%) | (N = 59) | | (N = 56) | | (N = 201) | | (N = 205) | |
| Any class | 41 | (69.5%) | 36 | (64.3%) | 122 | (60.7%) | 130 | (63.4%) |
| Infections and infestations | 19 | (32.2%) | 19 | (33.9%) | 61 | (30.3%) | 73 | (35.6%) |
| Nasopharyngitis | 12 | (20.3%) | 10 | (17.9%) | 37 | (18.4%) | 52 | (25.4%) |
| Pharyngitis | 1 | (1.7%) | 3 | (5.4%) | 7 | (3.5%) | 2 | (1.0%) |
| Influenza | 0 | | 1 | (1.8%) | 7 | (3.5%) | 3 | (1.5%) |
| Metabolism and nutrition disorders | 2 | (3.4%) | 1 | (1.8%) | 8 | (4.0%) | 6 | (2.9%) |
| Decreased appetite | 0 | | 0 | | 7 | (3.5%) | 0 | |
| Nervous system disorders | 7 | (11.9%) | 6 | (10.7%) | 19 | (9.5%) | 19 | (9.3%) |
| Dizziness | 4 | (6.8%) | 0 | | 4 | (2.0%) | 7 | (3.4%) |
| Headache | 1 | (1.7%) | 3 | (5.4%) | 5 | (2.5%) | 4 | (2.0%) |
| Gastrointestinal disorders | 13 | (22.0%) | 7 | (12.5%) | 55 | (27.4%) | 32 | (15.6%) |
| Nausea | 3 | (5.1%) | 2 | (3.6%) | 22 | (10.9%) | 3 | (1.5%) |
| Diarrhoea | 1 | (1.7%) | 0 | | 11 | (5.5%) | 6 | (2.9%) |
| Abdominal discomfort | 3 | (5.1%) | 0 | | 8 | (4.0%) | 0 | |
| Constipation | 3 | (5.1%) | 0 | | 6 | (3.0%) | 3 | (1.5%) |
| Dyspepsia | 3 | (5.1%) | 0 | | 3 | (1.5%) | 1 | (0.5%) |
| Dental caries | 0 | | 1 | (1.8%) | 4 | (2.0%) | 5 | (2.4%) |
| Skin and subcutaneous tissue disorders | 6 | (10.2%) | 5 | (8.9%) | 9 | (4.5%) | 20 | (9.8%) |
| Hyperhidrosis | 2 | (3.4%) | 1 | (1.8%) | 1 | (0.5%) | 6 | (2.9%) |
| Musculoskeletal and connective tissue | 7 | (11.9%) | 7 | (12.5%) | 28 | (13.9%) | 25 | (12.2%) |
| disorders | | | | | | | | |
| Back pain | 3 | (5.1%) | 3 | (5.4%) | 8 | (4.0%) | 6 | (2.9%) |
| General disorders and administration site | 10 | (16.9%) | 3 | (5.4%) | 21 | (10.4%) | 15 | (7.3%) |
| conditions | | | | | | | | |

| | | | | | | |
|---|---|---|---|---|---|---|
| Fatigue | 0 | 0 | 6 | (3.0%) | 4 | (2.0%) |

SGLT2 = Sodium glucose co-transporter 2,
TEAE: Treatment emergent adverse event,
SOC: System organ class,
PT: Preferred term.
MedDRA20.1.
n (%) = number and percentage of patients with at least one TEAE.
Note:
Table sorted by SOC internationally agreed order and decreasing frequency of PT sorted by decreasing frequency within a SOC according to all TEAE summary.
Only SOC with at least one PT ≥2% in at least one treatment group are presented.

TABLE 8
Summary of documented symptomatic hypoglycemia recorded on the dedicated
eCRF page and meeting protocol definition by SGLT2 inhibitor use at screening
during the on-treatment period-Safety population (EFC14114)

| | SGLT2 inhibitor use at screening | | SGLT2 inhibitor use at screening | |
|---|---|---|---|---|
| | Yes | | No | |
| | (N = 115) | | (N = 406) | |
| | Fixed Ratio | Insulin | Fixed Ratio | Insulin |
| | Combination | glargine | Combination | glargine |
| | (N = 59) | (N = 56) | (N = 201) | (N = 205) |
| Total patient years of exposure | 29.4 | 27.3 | 97.9 | 102.4 |
| Documented symptomatic hypoglycemia | | | | |
| (plasma glucose ≤70 mg/dL | | | | |
| [3.9 mmol/L]) | | | | |
| Number of patients with events, | 8 (13.6%) | 3 (5.4%) | 29 (14.4%) | 29 (14.1%) |
| n (%) | | | | |
| Number of patients with events per | 0.27 | 0.11 | 0.30 | 0.28 |
| patient yeara | | | | |
| Number of events | 15 | 8 | 78 | 62 |
| Number of events per patient yearb | 0.51 | 0.29 | 0.80 | 0.61 |

SGLT2 = Sodium glucose co-transporter 2,
IMP = Investigational medicinal product,
eCRF = Electronic case report form.
Total patient years of exposure: calculated as the time from the first injection of open-label IMP up to 1 day after the last injection of open-label IMP.
aCalculated as number of patients with events divided by total patient years of exposure.
bCalculated as number of events divided by total patient years of exposure.
Any hypoglycemia = Hypoglycemia recorded on the dedicated eCRF and meeting protocol definition for severe hypoglycemia, documented symptomatic hypoglycemia or asymptomatic hypoglycemia.
The on-treatment period is defined as the time from the first injection of open-label IMP up to 1 day after the last injection of open-label IMP, regardless of the introduction of rescue therapy.

1. A pharmaceutical combination, comprising
(a) a pharmaceutical formulation comprising
(i) lixisenatide or a pharmaceutically acceptable salt thereof, and
(ii) insulin glargine or a pharmaceutically acceptable salt thereof, and
(b) an SGLT2 inhibitor or a pharmaceutically acceptable salt thereof.

2. The pharmaceutical combination according to claim 1, further comprising (c) metformin or pharmaceutically acceptable salt thereof.

3. The pharmaceutical combination according to claim 1, wherein the SGLT2 inhibitor is empagliflozin, canagliflozin, dapagliflozin or ertugliflozin.

4. The pharmaceutical combination according to claim 1, wherein the pharmaceutical formulation (a) comprises insulin glargine in a concentration of 100 to 500 U/mL.

5. The pharmaceutical combination according to claim 1, wherein the pharmaceutical formulation (a) comprises insulin glargine in a concentration of 100 U/mL.

6. The pharmaceutical combination according to claim 1, wherein the pharmaceutical formulation (a) comprises lixisenatide in a concentration of 20 to 150 μg/ml.

7. The pharmaceutical combination according to claim 1, wherein the pharmaceutical formulation (a) comprises lixisenatide in a concentration of 33 μg/mL, 50 μg/mL or 100 μg/mL.

8. The pharmaceutical combination according to claim 1, wherein the pharmaceutical formulation (a) comprises insulin glargine in a concentration of 100 U/ml, and lixisenatide in a concentration of 33 μg/mL, 50 μg/mL or 100 μg/mL.

9. The pharmaceutical combination according to claim 1, for use in the treatment of a type 2 diabetes mellitus patient.

10. The pharmaceutical combination for use according to claim 9, wherein the type diabetes mellitus to be treated is not adequately controlled with the SGLT2 inhibitor alone.

11. The pharmaceutical combination for use according to claim 9, wherein the type 2 diabetes mellitus to be treated is not adequately controlled with an SGLT2 inhibitor and an oral anti-diabetic alone.

12. The pharmaceutical combination for use according to claim 9, wherein the type 2 diabetes mellitus to be treated is not adequately controlled with the SGLT2 inhibitor and metformin alone, or with an SGLT2 inhibitor and a GLP-1 receptor agonist alone.

13. The pharmaceutical combination for use according to claim 9, wherein the type 2 diabetes mellitus to be treated is not adequately controlled with the SGLT2 inhibitor, metformin and a GLP-1 receptor agonist alone.

14. The pharmaceutical combination for use according to claim 12, wherein the GLP-1 receptor agonist is selected from lixisenatide, exenatide, dulaglutide, and liraglutide.

15. The pharmaceutical combination for use according to claim 9, wherein the patient to be treated is obese.

16. The pharmaceutical combination for use according to claim 13, wherein the GLP-1 receptor agonist is selected from lixisenatide, exenatide, dulaglutide, and liraglutide.
